(19) United States
(12) Patent Application Publication
(10) Pub. No.: US 2026/0286346 A1
(43) Pub. Date: Sep. 24, 2026

(54) METHODS AND SYSTEMS FOR METHYLATION SEQUENCING

(71) Applicant: Freenome Holdings, Inc., South San Francisco, CA (US)

(72) Inventors: Eric Anthony ARIAZI, South San Francisco, CA (US); Grant Parker FLOWERS, South San Francisco, CA (US); Lourdes GOMEZ, South San Francisco, CA (US); Michael PEARSON, South San Francisco, CA (US)

(21) Appl. No.: 19/566,168

(22) Filed: Mar. 13, 2026

Related U.S. Application Data
(63) Continuation of application No. PCT/US2024/046649, filed on Sep. 13, 2024.
(60) Provisional application No. 63/582,948, filed on Sep. 15, 2023.
(60) Provisional application No. 63/644,275, filed on May 8, 2024.

(51) Int. Cl. C12N 15/10

(52) U.S. Cl. CPC C12N 15/1093

(57) ABSTRACT

Methods and systems provided herein address limitations of nucleic acid methylation sequencing and uses thereof for detection of disease by improving the quality, sensitivity, and accuracy. Methods provided herein further include minimally-destructive methylation conversion methods and nucleic acid library preparation techniques that improve methylation sequencing by minimizing signal loss and biases. Further provided herein are greater accuracy and more complete methylation state information, which permits higher quality feature generation for use in machine learning models and classifier generation.

### CROSS-REFERENCE

This application is a continuation of International Application No. PCT/US2024/046649, filed Sep. 13, 2024, which claims the benefit of U.S. Provisional Application No. 63/582,948, filed Sep. 15, 2023, and U.S. Provisional Application No. 63/644,275, filed May 8, 2024, each of which is entirely incorporated herein by reference for all purposes.

### BACKGROUND

Nucleic acid methylation can represent tumor characteristics and phenotypic states, and therefore, may have high potential for use in early disease detection and/or diagnosis as well as personalized medicine. For example, DNA methylation abnormalities may be associated with various stages of cancer, from tumor initiation to cancer progression and metastasis. Aberrant DNA methylation patterns may occur early in the pathogenesis of cancer, and can therefore provide a mechanism for early cancer detection. These properties enable the use of DNA methylation patterns for cancer diagnosis.

### SUMMARY

The methods and systems for nucleic acid library preparation for methylation sequencing provided herein address limitations of standard methylation sequencing methods by minimizing signal loss and reducing biases introduced in standard library preparation methods. These methods and systems thereby improve the quality and accuracy of nucleic acid methylation sequencing and uses thereof, for example, in detection of disease. More accurate and complete information regarding methylation state permits higher quality feature generation for use in machine learning models and classifier generation.

In an aspect, the present disclosure provides a method of preparing a sequencing library for methylation sequencing of one or more nucleic acid molecules of a biological sample or derivative thereof, comprising:
(a) obtaining a nucleic acid composition, wherein the nucleic acid composition comprises a plurality of single-stranded nucleic acid molecules obtained or derived from the biological sample;
(b) ligating a nucleic acid adapter to a single-stranded nucleic acid molecule of the plurality of single-stranded nucleic acid molecules to generate an adapter-ligated nucleic acid molecule, wherein the nucleic acid adapter comprises nucleic acids that are resistant to base conversion by a methylation enrichment method; and
(c) subjecting the adapter-ligated nucleic acid molecule to conditions sufficient to convert unmethylated cytosines to uracils using a methylation enrichment method, thereby generating a converted adapter-ligated nucleic acid molecule.

In some embodiments, the ligating in (b) further comprises treating with a deoxyribonucleic acid (DNA) ligase. In some embodiments, the ligating in (b) further comprises treating with a polynucleotide kinase.

In some embodiments, the nucleic acid adapter comprises a double stranded oligonucleotide comprising an adapter sequence and an overhang sequence. In some embodiments, the overhang sequence is a 3′ overhang sequence. In some embodiments, the overhang sequence comprises a random sequence of oligonucleotides.

In some embodiments, the nucleic acid adapter comprises one or more methylated cytosine bases. In some embodiments, the nucleic acid adapter does not comprise methylated cytosine bases or unmethylated cytosine bases.

In some embodiments, the nucleic acid adapter comprises a unique molecular identifier. In some embodiments, the unique molecular identifier is configured to enable measurement of an enrichment efficiency of the methylation conversion method.

In some embodiments, the method further comprises, prior to (a), processing the biological sample or derivative thereof to generate the plurality of single-stranded nucleic acid molecules. In some embodiments, the processing comprises denaturing a double-stranded nucleic acid molecule in the biological sample or derivative thereof. In some embodiments, the denaturing further comprises applying heat to the double-stranded nucleic acid molecule. In some embodiments, the denaturing further comprises applying heat to the double-stranded nucleic acid molecule, and then performing rapid cooling of the denatured single stranded nucleic acid molecule.

In some embodiments, the method further comprises treating the single-stranded nucleic acid molecule or derivative thereof with a binding agent configured to reduce a likelihood of formation of nucleic acid duplexes. In some embodiments, the method does not comprise treating the single-stranded nucleic acid molecule or derivative thereof with a binding agent configured to reduce a likelihood of formation of nucleic acid duplexes. In some embodiments, the method the binding agent is a single-stranded nucleic acid binding protein (SSB).

In some embodiments, the method further comprises amplifying the converted adapter-ligated nucleic acid molecule. In some embodiments, the amplifying comprises polymerase chain reaction (PCR).

In some embodiments, the method further comprises contacting the converted adapter-ligated nucleic acid molecule or derivative thereof with nucleic acid probes to generate an enriched nucleic acid molecule, wherein the nucleic acid probes comprise a nucleic acid sequence that is at least partially complementary to CpG or CH loci of a reference panel. In some embodiments, the nucleic acid probes comprise unmethylated nucleic acid probes. In some embodiments, the nucleic acid probes are configured to selectively hybridize to one or more target regions of interest that correspond to unmethylated cytosine bases at a CpG locus from the CpG or CH loci of the reference panel. In some embodiments, the nucleic acid probes are configured to selectively hybridize to one or more target regions of interest that correspond to methylated cytosine bases at a CpG locus from the CpG or CH loci of the reference panel.

In some embodiments, the method further comprises determining a nucleic acid sequence of the enriched nucleic acid molecule or derivative thereof.

In some embodiments, the method further comprises sequencing the enriched nucleic acid molecule or derivative thereof to generate sequencing data. In some embodiments, the method further comprises analyzing the sequencing data to generate a methylation profile of the nucleic acid molecule of the biological sample or derivative thereof. In some embodiments, the methylation profile comprises hypermethylation and/or hypomethylation analysis. In other embodiments, the methylation profile comprises hypermethylation analysis. In still other embodiments, the methylation profile comprises hypomethylation analysis. In some embodiments, the analyzing further comprises comparing the sequencing data to a reference sequence.

In some embodiments, the method further comprises, after (b), subjecting the adapter-ligated nucleic acid molecule to an extension reaction to generate a partially double-stranded nucleic acid molecule or a fully double-stranded nucleic acid molecule. In some embodiments, the extension reaction is performed in the presence of a polymerase, a plurality of deoxynucleotide triphosphates (dNTPs), and a primer complementary to a 3′ end of the nucleic acid adapter.

In some embodiments, the nucleic acid molecule is deoxyribonucleic acid (DNA). In some embodiments, the DNA is cell-free DNA.

In some embodiments, the biological sample is a cell-free biological sample. In some embodiments, the cell-free biological sample is a plasma sample.

In some embodiments, the methylation enrichment method comprises treatment with one or more enzymes. In some embodiments, the methylation enrichment method comprises treatment with a ten eleven translocation (TET) enzyme. In some embodiments, the methylation enrichment method does not comprise treatment with bisulfite.

In an aspect, the present disclosure provides a method of preparing a sequencing library for methylation sequencing of a nucleic acid molecule of a biological sample or derivative thereof, comprising:
(a) obtaining a nucleic acid composition, wherein the nucleic acid composition comprises a plurality of single-stranded nucleic acid molecules obtained or derived from the biological sample;
(b) subjecting a single-stranded nucleic acid molecule of the plurality of single-stranded nucleic acid molecules to conditions sufficient to convert unmethylated cytosines to uracils using a methylation enrichment method, thereby generating a converted single-stranded nucleic acid molecule; and
(c) ligating a nucleic acid adapter to the converted single-stranded nucleic acid molecule to generate an adapter-ligated converted nucleic acid molecule, wherein the nucleic acid adapter comprises nucleic acids that are resistant to base conversion by the methylation enrichment method.

In some embodiments, the ligating in (c) further comprises treating with a deoxyribonucleic acid (DNA) ligase. In some embodiments, the ligating in (c) further comprises treating with a polynucleotide kinase.

In some embodiments, the nucleic acid adapter comprises a double stranded oligonucleotide comprising an adapter sequence and an overhang sequence. In some embodiments, the overhang sequence is a 3′ overhang sequence. In some embodiments, the overhang sequence comprises a random sequence of oligonucleotides.

In some embodiments, the nucleic acid adapter comprises one or more methylated cytosine bases. In some embodiments, the nucleic acid adapter does not comprise methylated cytosine bases or unmethylated cytosine bases.

In some embodiments, the nucleic acid adapter comprises a unique molecular identifier. In some embodiments, the unique molecular identifier is configured to enable measurement of an enrichment efficiency of the methylation enrichment method.

In some embodiments, the method further comprises, prior to (a), processing the biological sample or derivative thereof to generate the plurality of single-stranded nucleic acid molecules. In some embodiments, the processing comprises denaturing a double-stranded nucleic acid molecule in the biological sample or derivative thereof. In some embodiments, the denaturing comprises applying heat to the double-stranded nucleic acid molecule. In some embodiments, the denaturing comprises applying heat to the double-stranded nucleic acid molecule to produce the plurality of single-stranded nucleic acid molecules, and then performing rapid cooling on the plurality of single-stranded nucleic acid molecules.

In some embodiments, the method further comprises treating the single-stranded nucleic acid molecule or derivative thereof with a binding agent configured to reduce a likelihood of formation of nucleic acid duplexes. In some embodiments, the method does not comprise treating the single-stranded nucleic acid molecule or derivative thereof with a binding agent configured to reduce a likelihood of formation of nucleic acid duplexes. In some embodiments, the binding agent is a single-stranded nucleic acid binding protein (SSB).

In some embodiments, the method further comprises amplifying the adapter-ligated converted nucleic acid molecule. In some embodiments, the amplifying comprises polymerase chain reaction (PCR).

In some embodiments, the method further comprises contacting the adapter-ligated converted nucleic acid molecule or derivative thereof with nucleic acid probes to generate an enriched nucleic acid molecule, wherein the nucleic acid probes comprise a nucleic acid sequence that is at least partially complementary to CpG or CH loci of a reference panel. In some embodiments, the nucleic acid probes comprise unmethylated nucleic acid probes. In some embodiments, the nucleic acid probes are configured to selectively hybridize to one or more target regions of interest that correspond to unmethylated cytosine bases at a CpG locus from the CpG or CH loci of the reference panel. In some embodiments, the nucleic acid probes are configured to selectively hybridize to one or more target regions of interest that correspond to methylated cytosine bases at a CpG locus from the CpG or CH loci of the reference panel.

In some embodiments, the method further comprises determining a nucleic acid sequence of the enriched nucleic acid molecule or derivative thereof. In some embodiments, the method further comprises sequencing the enriched nucleic acid molecule or derivative thereof to generate sequencing data. In some embodiments, the method further comprises analyzing the sequencing data to generate a methylation profile of the nucleic acid molecule of the biological sample or derivative thereof. In some embodiments, the methylation profile comprises hypermethylation and/or hypomethylation analysis. In other embodiments, the methylation profile comprises hypermethylation analysis. In still other embodiments, the methylation profile comprises hypomethylation analysis. In some embodiments, the method for analyzing further comprises comparing the sequencing data to a reference sequence.

In some embodiments, the method further comprises, after (b), subjecting the adapter-ligated converted nucleic acid molecule to an extension reaction to generate a partially double-stranded nucleic acid molecule or a fully double-stranded nucleic acid molecule. In some embodiments, the extension reaction is performed in the presence of a polymerase, a plurality of deoxynucleotide triphosphates (dNTPs), and a primer complementary to a 3′ end of the nucleic acid adapter.

In some embodiments, the nucleic acid molecule is deoxyribonucleic acid (DNA). In some embodiments, the DNA is cell-free DNA.

In some embodiments, the biological sample is a cell-free biological sample. In some embodiments, the cell-free biological sample is a plasma sample.

In some embodiments, the methylation enrichment method comprises treatment with one or more enzymes. In some embodiments, the methylation enrichment method comprises treatment with a ten eleven translocation (TET) enzyme. In some embodiments, the methylation enrichment method does not comprise treatment with bisulfite.

In an aspect, the present disclosure provides a method comprising:
(a) ligating a nucleic acid adapter to a single-stranded nucleic acid molecule obtained or derived from a biological sample of a subject to generate an adapter-ligated nucleic acid molecule, wherein the nucleic acid adapter comprises nucleic acids that are resistant to base conversion by a methylation enrichment method; and
(b) subjecting the adapter-ligated nucleic acid molecule to conditions sufficient to convert unmethylated cytosines to uracils using a methylation enrichment method, thereby generating a converted adapter-ligated nucleic acid molecule;
(c) amplifying the converted adapter-ligated nucleic acid molecule to generate an amplified nucleic acid molecule;
(d) contacting the amplified nucleic acid molecule or derivative thereof with nucleic acid probes to generate an enriched nucleic acid molecule, wherein the nucleic acid probes comprise a nucleic acid sequence that is at least partially complementary to CpG or CH loci of a reference panel;
(e) determining a nucleic acid sequence of the enriched nucleic acid molecule or derivative thereof;
(f) comparing the nucleic acid sequence of the enriched nucleic acid molecule or derivative thereof to a reference nucleic acid sequence; and
(g) training a machine learning model to produce a classifier that distinguishes between subjects having a cancer and subjects not having the cancer, wherein the machine learning model is trained with methylation profiles generated from (i) a first set of nucleic acid samples from subjects having the cancer and (ii) a second set of nucleic acid samples from subjects not having the cancer.

In some embodiments, the methylation profile comprises hypermethylation and/or hypomethylation analysis.

In other embodiments, the methylation profile comprises hypermethylation analysis.

In still other embodiments, the methylation profile comprises hypomethylation analysis.

In some embodiments, the amplifying comprises a polymerase chain reaction (PCR).

In some embodiments, the method further comprises determining the nucleic acid sequence of the enriched nucleic acid molecule or derivative thereof at a depth of >100×.

In some embodiments, the nucleic acid probes comprise unmethylated nucleic acids. In some embodiments, the nucleic acid probes are configured to selectively hybridize to one or more target regions of interest that correspond to unmethylated cytosine bases at a CpG locus from the CpG or CH loci of the reference panel. In some embodiments, the nucleic acid probes are configured to selectively hybridize to one or more target regions of interest that correspond to methylated cytosine bases at a CpG locus from the CpG or CH loci of the reference panel.

In some embodiments, the method further comprises sequencing the enriched nucleic acid molecule or derivative thereof to generate sequencing data. In some embodiments, the method further comprises analyzing the sequencing data to generate a methylation profile of the nucleic acid molecule of the biological sample or derivative thereof. In some embodiments, the methylation profile comprises hypermethylation and/or hypomethylation analysis. In other embodiments, the methylation profile comprises hypermethylation analysis. In still other embodiments, the methylation profile comprises hypomethylation analysis.

In some embodiments, the method further comprises, after (a), subjecting the adapter-ligated converted nucleic acid molecule to an extension reaction to generate a partially double-stranded nucleic acid molecule or fully double-stranded nucleic acid molecule. In some embodiments, the extension reaction is performed in the presence of a polymerase, a plurality of deoxynucleotide triphosphates (dNTPs), and a primer complementary to a 3′ end of the nucleic acid adapter.

In some embodiments, the single-stranded nucleic acid molecule is deoxyribonucleic acid (DNA). In some embodiments, the DNA is cell-free DNA.

In some embodiments, the biological sample is a cell-free biological sample. In some embodiments, the cell-free biological sample is a plasma sample.

In some embodiments, the methylation enrichment method comprises treatment with one or more enzymes. In some embodiments, the methylation enrichment method comprises treatment with a ten eleven translocation (TET) enzyme. In some embodiments, the methylation enrichment method does not comprise treatment with bisulfite.

In some embodiments, the reference panel comprises CpG or CH loci associated with transcription start sites.

In some embodiments, the method further comprises identifying a tissue-of-origin for the nucleic acid molecule.

In some embodiments, the method further comprises identifying a genomic position and a fragment length for the nucleic acid molecule.

In some embodiments, the machine learning model is trained using a feature input selected from the group consisting of: base wise methylation % for CpG, base wise methylation % for CHG, base wise methylation % for CHH, the count or rate of observing fragments with different counts or rates of methylated CpGs in a region, conversion efficiency, hypomethylated blocks, methylation levels for CPG, methylation levels for CHH, methylation levels for CHG, fragment length, fragment midpoint, methylation levels for chrM, methylation levels for LINE1, methylation levels for ALU, dinucleotide coverage, evenness of coverage, mean CpG coverage globally, mean coverage at CpG islands, CGI shelves, and CGI shores.

In some embodiments, the classifier that distinguishes between subjects having the cancer and subjects not having the cancer: sets of measured values representative of methylation profiles from methylation sequencing data from subjects having a cancer and subjects not having the cancer, wherein the measured values are used to generate a set of features corresponding to properties of the methylation profiles, wherein the set of features are processed by a machine learning or statistical model, wherein the machine learning or statistical model provides a feature vector useful as a classifier that distinguishes the population of subjects having the cancer and subjects not having the cancer.

In some embodiments, the method further comprises:
(a) assaying by the methylation enrichment method to obtain a methylation profile of the biological sample;
(b) classifying by a trained machine learning algorithm the methylation profile of the biological sample as indicative of a presence of the cancer in the subject; and
(c) outputting a report that identifies the biological sample as negative for the cancer if the trained machine learning algorithm classifies the biological sample as negative for the cancer at a specified confidence level.

In some embodiments, the methylation profile comprises hypermethylation and/or hypomethylation analysis.

In other embodiments, the methylation profile comprises hypermethylation analysis.

In still other embodiments, the methylation profile comprises hypomethylation analysis.

In some embodiments, the method further comprises:
(a) determining a baseline methylation profile of the biological sample of the subject at a baseline methylation state;
(b) determining a test methylation profile of a biological sample of the subject at one or more time points following the baseline methylation state; and
(c) determining a change in the test methylation profile as compared to the baseline methylation profile, wherein the change indicates a change in a minimal residual disease status of the subject.

In some embodiments, the methylation profile comprises hypermethylation and/or hypomethylation analysis.

In other embodiments, the methylation profile comprises hypermethylation analysis.

In still other embodiments, the methylation profile comprises hypomethylation analysis.

In some embodiments, the cancer comprises two or more of colorectal cancer, breast cancer, pancreatic cancer, liver, or lung cancer.

In other embodiments, the cancer is colorectal cancer, breast cancer, pancreatic cancer, liver, or lung cancer.

In one embodiment, the cancer is colorectal cancer. In another embodiment, the cancer is lung cancer. In still another embodiment, the cancer is pancreatic cancer. In yet another embodiment, the cancer is liver cancer.

In some embodiments, the minimal residual disease status is selected from the group consisting of response to a treatment, tumor load, residual tumor post-surgery, relapse, secondary screen, primary screen, and cancer progression.

In an aspect, the present disclosure provides a method, comprising: ligating a nucleic acid adapter to a single-stranded nucleic acid molecule obtained or derived from a biological sample of a subject to generate an adapter-ligated nucleic acid molecule, wherein the nucleic acid adapter comprises nucleic acids that are resistant to base conversion by a methylation enrichment method; subjecting the adapter-ligated nucleic acid molecule to conditions sufficient to convert unmethylated cytosines to uracils using a methylation enrichment method, thereby generating a converted adapter-ligated nucleic acid molecule; amplifying the converted adapter-ligated nucleic acid molecule to generate an amplified nucleic acid molecule; contacting the amplified nucleic acid molecule or derivative thereof with nucleic acid probes to generate an enriched nucleic acid molecule, wherein the nucleic acid probes comprise a nucleic acid sequence that is at least partially complementary to CpG or CH loci of a reference panel; determining a nucleic acid sequence of the enriched nucleic acid molecule or derivative thereof; comparing the nucleic acid sequence of the enriched nucleic acid molecule or derivative thereof to a reference nucleic acid sequence; and training a machine learning model to produce a classifier that distinguishes between subjects having an indication and subjects not having the indication, wherein the machine learning model is trained with methylation profiles generated from (i) a first set of nucleic acid samples from subjects having the indication; and (ii) a second set of nucleic acid samples from subjects not having the indication.

In some embodiments, the method further comprises assaying by the methylation enrichment method to obtain a methylation profile of the biological sample; classifying by a trained machine learning algorithm the methylation profile of the biological sample as indicative of a presence of the indication in the subject; and outputting a report that identifies the biological sample as negative for the indication if the trained machine learning algorithm classifies the biological sample as negative for the indication at a specified confidence level.

In some embodiments, the method further comprises determining a baseline methylation profile of the biological sample of the subject at a baseline methylation state; determining a test methylation profile of a biological sample of the subject at one or more time points following the baseline methylation state; and determining a change in the test methylation profile as compared to the baseline methylation profile, wherein the change indicates a change in a minimal residual disease status of the indication in the subject.

In some embodiments, the minimal residual disease status is selected from the group consisting of: response to a treatment, relapse, secondary screen, primary screen, and indication progression.

In some embodiments, the methylation profile comprises hypermethylation analysis and/or hypomethylation analysis.

In some embodiments, the indication comprises gut-associated diseases, immune-mediated inflammatory diseases, neurological diseases, kidney diseases, prenatal diseases, or metabolic diseases.

In an aspect, the present disclosure provides a system comprising:
(a) a computer readable medium product comprising a classifier of the present disclosure,
   wherein the classifier comprises: a set of measured values representative of methylation profiles from methylation sequencing data from subjects having a cancer and subjects not having the cancer, wherein the set of measured values is used to generate a set of features corresponding to properties of the methylation profiles from subjects having a cancer and subjects not having the cancer, wherein the set of features is processed by a machine learning or statistical model, wherein the machine learning or statistical model provides a feature vector useful for distinguishing subjects having the cancer and subjects not having the cancer; and
(b) one or more processors for executing instructions stored on the computer readable medium product.

In some embodiments, the classifier is selected from the group consisting of a linear discriminant analysis (LDA) classifier, a quadratic discriminant analysis (QDA) classifier, a support vector machine (SVM) classifier, a random forest (RF) classifier, a linear kernel support vector machine classifier, a first order polynomial kernel support vector machine classifier, a second order polynomial kernel support vector machine classifier, a ridge regression classifier, an elastic net algorithm classifier, a sequential minimal optimization algorithm classifier, a naive Bayes algorithm classifier, and a non-negative matrix factorization (NMF) predictor algorithm classifier.

In some embodiments, the system is further configured to perform any of the above methods.

In some embodiments, the system comprises one or more processors configured to perform any of the above methods.

In some embodiments, the system comprises modules that respectively perform the operations of any of the above methods.

In an aspect, the present disclosure provides a kit for detecting a cancer comprising reagents for performing any of the above methods, and instructions for detecting a cancer signal.

In some embodiments, the reagents are selected from the group consisting of primer sets, PCR reaction components, sequencing reagents, methylation enrichment reagents, and library preparation reagents.

In some embodiments, the machine learning model is trained using training data obtained from training biological samples, a first subset of the training biological samples identified as corresponding to a subject having a cell proliferative disorder and a second subset of the training biological samples identified corresponding to a subject as not having the cell proliferative disorder.

In some embodiments, the classifier is provided in a system for detecting a cell proliferative disorder, the system comprising: a) a computer-readable medium comprising a classifier operable to classify the subjects based on a methylation signature panel; and b) one or more processors for executing instructions stored on the computer-readable medium.

In some embodiments, the system comprises a classification circuit that is configured as a machine learning classifier selected from the group consisting of a deep learning classifier, a neural network classifier, a linear discriminant analysis (LDA) classifier, a quadratic discriminant analysis (QDA) classifier, a support vector machine (SVM) classifier, a random forest (RF) classifier, K nearest neighbor, a linear kernel support vector machine classifier, a first or second order polynomial kernel support vector machine classifier, a ridge regression classifier, an elastic net algorithm classifier, a sequential minimal optimization algorithm classifier, a naive Bayes algorithm classifier, and principal component analysis classifier.

In some embodiments, the method further comprises presenting a report or a graphical user interface of an electronic device of a user. In some embodiments, the user is the subject, individual, or patient.

In some embodiments, the method further comprises determining a likelihood of the determination of a presence or susceptibility of cell proliferative disorder in the subject, individual, or patient.

In some embodiments, the trained algorithm (e.g., machine learning model or classifier) comprises a supervised or semi-supervised machine learning algorithm. In some embodiments, the supervised machine learning algorithm comprises a deep learning algorithm, a support vector machine (SVM), a neural network, or a random forest.

In some embodiments, the method further comprises providing the subject with a second diagnostic assay or procedure on the subject based at least in part on the methylation signature profile or analysis describe herein, such as a non-invasive detection assay or procedure including, but not limited to, a colonoscopy, a CT-scan, an MRI, an ultrasound, or other procedures.

In some embodiments, the method further comprises providing the subject with a therapeutic intervention or administering a treatment to the subject based at least in part on the methylation signature profile or analysis described herein, such as a therapeutic intervention to treat a patient with the cell proliferative disorder (e.g., chemotherapy, radiotherapy, immunotherapy, or surgery).

In some embodiments, the method further comprises monitoring the presence or susceptibility of the cell proliferative disorder, wherein the monitoring comprises assessing the presence or susceptibility of the cell proliferative disorder of the subject at a plurality of time points, wherein the assessing is based at least on the presence or susceptibility of the cell proliferative disorder determined at each of the plurality of time points.

In some embodiments, a difference in the assessment of the presence or susceptibility of the cell proliferative disorder of the subject among the plurality of time points is indicative of one or more clinical indications selected from the group consisting of: (i) a diagnosis of the presence or susceptibility of the cell proliferative disorder of the subject, (ii) a prognosis of the presence or susceptibility of the cell proliferative disorder of the subject, and (iii) an efficacy or non-efficacy of a course of treatment for treating the presence or susceptibility of the cell proliferative disorder of the subject.

In some embodiments, the method further comprises stratifying the cell proliferative disorder of the subject by using the trained algorithm to determine a sub-type of the cell proliferative disorder of the subject from among a plurality of distinct subtypes or stages of the cell proliferative disorder.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Another aspect of the present disclosure provides a system comprising: a) a computer-readable medium comprising a classifier for distinguishing a population of subjects having a cell proliferative disorder from subjects not having the cell proliferative disorder based on a methylation signature panel using a machine learning model; and b) one or more processors for executing instructions stored on the computer-readable medium.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent that publications and patents or patent applications incorporated by reference contradict the disclosure contained in the specification, the specification is intended to supersede and/or take precedence over any such contradictory material.

### BRIEF DESCRIPTION OF DRAWINGS

FIG. 1 provides a schematic of an example single-stranded DNA (ssDNA) library preparation method as described herein versus a standard double-stranded DNA (dsDNA) library preparation method.

FIG. 2 provides a schematic of an example ssDNA library preparation workflow.

FIG. 3 provides a comparison of examples of methylation signals relative to distance from the 3′ ends of hypermethylated DNA fragments using an ssDNA library preparation method versus a dsDNA library preparation method.

FIG. 4 provides a comparison of examples of captured reads relative to DNA fragment length detected using an ssDNA library preparation method versus a dsDNA library preparation method.

FIG. 5 provides a comparison of examples of hypermethylation scores of cell-free DNA (cfDNA) extracted from healthy/cancer-negative (NEG), advance adenoma (AA), and colorectal cancer (CRC) patient samples detected using an ssDNA library preparation method as described herein versus a dsDNA library preparation method.

FIG. 6 provides a comparison of examples of hypermethylation scores of cfDNA extracted from healthy/cancer-negative (NEG), liver cancer (Liver), lung cancer (Lung), and pancreatic cancer (Pancreas) patient samples detected using an ssDNA library preparation method as described herein versus a dsDNA library preparation method.

FIG. 7 provides a comparison of examples of hypomethylation scores of cfDNA extracted from healthy/cancer-negative (NEG), advance adenoma (AA), and colorectal cancer (CRC) patient samples detected using an ssDNA library preparation method as described herein versus a dsDNA library preparation method.

FIG. 8 provides a comparison of examples of hypomethylation scores of cfDNA extracted from healthy/cancer-negative (NEG), liver cancer (Liver), lung cancer (Lung), and pancreatic cancer (Pancreas) patient samples detected using an ssDNA library preparation method as described herein versus a dsDNA library preparation method.

FIGS. 9A-9B provide schematics of examples of two methods by which ssDNA libraries generated by the methods disclosed herein may be converted into dsDNA libraries after ligation. After a clean-up operation to remove excess adapters following adapter ligation, a single-stranded specific (SSS) primer may be annealed to the 3′ end of the adapter-ligated nucleic acid to initiate a primer extension reaction to generate a strand complementary to the adapter-ligated nucleic acid (FIG. 9A). Alternatively, incompletely overlapping splint adapters may be ligated to the ssDNA followed by annealing of a SSS primer to the 3′ end of the adapter-ligated nucleic acid to initiate a primer extension reaction and removal of excess adapters (FIG. 9B). This method allows nucleic acid amplification without requiring an initial clean-up operation to remove excess adapters.

FIG. 10 provides a comparison of an example of the protection rate of methylated cytosines (mC) in ssDNA using a ssDNA library workflow with second strand synthesis (SSS) and with no second strand synthesis (SOP) after converting ssDNA libraries to dsDNA libraries.

FIG. 11 shows an example of a computer system that is programmed or otherwise configured to implement methods provided herein.

### DETAILED DESCRIPTION

Provided herein are methods and systems relating to library preparation and sequencing of methylated regions for methylation profiling of nucleic acids, such as cell-free deoxyribonucleic acid (cfDNA). The methods and systems address limitations of existing library preparation and subsequent methylation sequencing and profiling of nucleic acids from a biological sample by minimizing signal loss, reducing biases, and improving coverage, uniformity of coverage, resolution, and accuracy of methylation data to support practical applications such as those described herein. The resulting sequencing data obtained from methods provided herein may be useful for applications that use methylation profiling data for classifying or stratifying a population of individuals. Such classifying or stratifying of a population of individuals may include, for example, identifying and/or detecting individuals as having a disease, staging disease progression (including detection of minimal residual disease (MRD) or determining an individual's response to a particular treatment for a disease.

Methylation analysis may be coupled with DNA sequencing to determine a likelihood that a sample is normal, tumor-derived, or disease-positive. For example, a relative abundance of methylated or unmethylated DNA fragments that map or align to specific genomic regions may be used to detect or determine disease likelihood. Such sequencing methods may include, for example, standard DNA library preparation in which enzymatic end repair processes are used prior to attaching sequencing adapters to double-stranded DNA (dsDNA) fragments. However, these processes may not preserve fragment ends and may not capture nicked or single-stranded DNA, thereby reducing the overall methylation signal and biasing the fragment length distribution. As a result, end repair may result in a relative undercounting of methylated molecules and an overcounting of unmethylated molecules, both of which may lead to an underestimation of the signal contribution from the tumor-derived DNA. In turn, the sensitivity of these methods for cancer detection may be reduced.

The methods and systems herein may comprise preparing a single-stranded DNA (ssDNA) sequencing library and subjecting a sample from the ssDNA library to methylation interrogation treatment method such that unmethylated cytosine bases in the ssDNA are converted to uracil bases. The methods may include denaturing double-stranded DNA (dsDNA) to ssDNA and attaching conversion-resistant nucleic acid adapters to the ssDNA, which permits evaluation of methylation in molecules in a way that does not require end-repair and results in an improved, more accurate methylation analysis that is not hindered by the undercounting of methylated molecules generated from dsDNA library preparation methods. After attaching the conversion-resistant nucleic acid adapters to the ssDNA, the adapter-ligated ssDNA may be subjected to enzymatic methyl (EM) conversion or bisulfite treatment methods to provide a base-level resolution of DNA methylation. In one embodiment, the adapter ligation is performed before methylation base conversion. In another embodiment, methylation interrogating methods are performed prior to adapter ligation. In certain cases, performing adapter ligation before methylation base conversion may be preferable to performing adapter ligation after methylation base conversion. Further, enzymatic conversion methods may be preferable to chemical conversion methods (e.g., bisulfite treatment) as chemical treatment may lead to more extensive DNA damage and molecular loss than enzymatic methods.

For methods in which ssDNA library preparation is coupled with EM conversion, adapter-ligated sequences may be amplified and sequenced after EM conversion. However, standard adapters may not be compatible with these methods. Thus, the library adapter sequences described in the methods herein may deviate from adapter sequences used for standard double-stranded sequencing library preparation with respect to the content and modifications of cytosine residues such that the adapter residues are resistant to conversion by either chemical or enzymatic conversion methods. Additionally, in certain embodiments, the library adapter sequences may include additional unique molecular identifier (UMI) sequences that may be used to monitor the efficiency of the EM conversion reactions.

Various ssDNA library preparation methods may be used in combination with methylation interrogation treatment methods, including, for example, adaptase-based ssDNA and Terminal deoxyribonucleotidyl transferase (TdT)-assisted Adenylate Connector-mediated SsDNA (TACS) library preparation. Additional ssDNA library preparation approaches may involve the use of T4-ligase mediated single-stranded nucleic acid ligation with splinted oligonucleotides. These approaches may include SPLinted Adapter Tagging (SPLAT) and Single Reaction Single-stranded LibrarY preparation (SRSLY) or Single strand Adapter Library Preparation (SALP), which may utilize a ssDNA binding protein (SSB) and eliminate end-repair to carry out adapter ligation.

Adaptase-based, TACS, and SPLAT methods of ssDNA library preparation methods may involve additional clean-up operations that are not found in splint-adapter based ssDNA library preparation methods. These clean-ups may be associated with DNA loss, especially of shorter DNA fragments that harbor methylation signals that are important for disease detection. These methods when coupled with methylation analysis methods, e.g., EM conversion or bisulfite treatment, which may precede library preparation, can lead to further signal loss. The purification and recovery processes for EM conversion and bisulfite treatment may result in greater DNA loss when shorter, adapter-free DNA is processed than when longer adapter-ligated DNA is processed. Further, as EM conversion or bisulfite treatment converts unmethylated cytosines within adapter sequences, numerous additional design considerations may be required for compatibility of these adapters with downstream processes (e.g., amplification and sequencing). These additional design constraints may also apply to adapters used for splint ligation processes, such as SPLAT or SRSLY, which may not intrinsically compatible with enzymatic conversion downstream of library preparation. Furthermore, addition of SSB treatment, e.g., in the SRSLY workflow, may be extraneous for the library preparation of cfDNA and may impede the performance of methylation conversion processes.

Methods described herein may include ssDNA library preparation methods that are compatible with methylation conversion workflows without the need for SSB treatment. The methods may be useful for determining the native methylation status of cfDNA by sequencing by minimizing molecular loss and removing biases introduced in standard library preparation methods. After denaturing dsDNA to ssDNA, ssDNA may be ligated to adapters that are designed to be insensitive to subsequent enzymatic or chemical treatment used for the detection of DNA methylation. These adapter sequences may also contain modules to assess the performance ore efficiency level of these enzymatic or chemical treatment methods. After treatment, the ligated, treated DNA may be subjected to amplification and sequencing. The information obtained from this sequencing, including but not limited to, the identity, quantity, size, precise ends, and relative methylation status of the DNA fragments may be used to determine whether the cfDNA is cancer-derived.

Compared to existing library preparation methods coupled with methyl conversion methods, the methods described herein provide various advantages. The methods more faithfully preserve the methylation signal from DNA extraction to library preparation to methyl conversion to sequencing than existing methods. For example, the end repair process of dsDNA library preparation both excises and inserts DNA from 3′ and 5′ overhangs, respectively. The consequence of these modifications is an underestimation of true DNA methylation in sequencing of dsDNA EM-converted libraries. As DNA fragment methylation status alone is sufficient to identify cancer-patient-derived cfDNA, this impaired fidelity reduces the sensitivity of this method of cancer detection. Similarly, the reduced methylation read-out in sequenced dsDNA, EM-converted libraries reduce the utility of low or absent DNA methylation as a signal for cancer detection. Both of these issues are overcome using ssDNA library preparation and EM conversion.

The method may permit the sequencing of DNA that is normally lost in dsDNA library preparation including short ssDNA and single-stranded fragments from nicked dsDNA. The identities and relative quantities of these fragments may be a tool in the discrimination of cancer patient-derived DNA from healthy patient-derived DNA.

The library preparation methods herein preserve ends of DNA fragments that may be lost or altered in an end repair process required for traditional dsDNA library preparation methods. Fragment-end data may be a source of signal used to identify tumor-derived DNA.

Existing ssDNA library preparation methods must capture DNA fragments after bisulfite or EM conversion in order to preserve adapter sequences for downstream processing (e.g., sequencing). As these DNA samples must undergo clean-up processes between conversion and adapter ligation, the samples may be subjected to more extensive DNA loss than the methods described herein, in which larger, adapter-ligated DNA molecules are first cleaned after ligation. The process improves molecular recovery and thereby improves the ability of this assay, particularly to smaller fractions of tumor-derived signal within a population of cell-free DNA. The methods herein may also remove extraneous reagents such as SSB treatment, which may impair downstream assay performance.

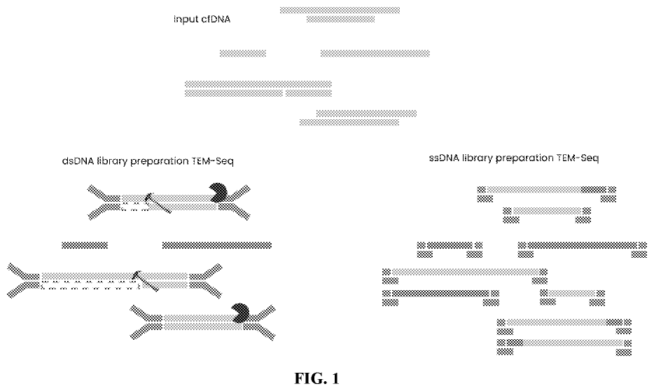

FIG. 1 provides a schematic of an example of dsDNA library preparation versus ssDNA library preparation as described in the methods herein. In this example, input cfDNA may be diverse with respect to strandedness (e.g., single stranded or double stranded), presence of nicks, and the presence and lengths of 5′ and 3′ overhangs.

As illustrated in FIG. 1, ssDNA library preparation methods may preserve fragment ends and capture DNA that is not captured using standard dsDNA library preparation methods. In the dsDNA library preparation workflow (FIG. 1, bottom left), end-repairing enzymes are used to remove or extend ends of dsDNA fragments (pink open boxes) before Y-shaped adapters are ligated onto the dsDNA. These end repair processes may result in the exclusion of methylation signal from these fragment ends. In the ssDNA library preparation workflow (FIG. 1, bottom right), conversion-tolerant ssDNA-compatible adapters are directly ligated to the ssDNA fragments. The end repair processes are excluded in the ssDNA library preparation workflow, thereby preserving fragment ends and methylation signals arising therefrom. As described herein, the ssDNA library preparation methods can therefore capture essentially all input DNA (after denaturing of dsDNA), including fragments and regions of DNA that are lost in dsDNA end-repair and library preparation (pink lines). Additionally, the ssDNA library preparation methods described herein capture native DNA methylation, while excluding end-repair artifacts resulting from dsDNA library preparation methods.

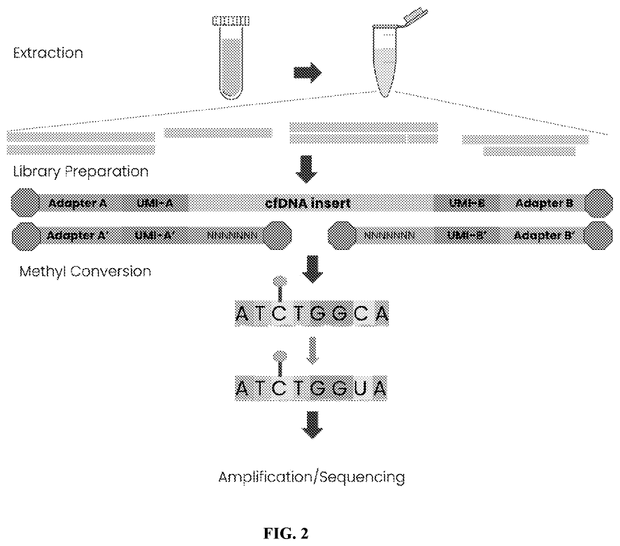

FIG. 2 provides a schematic of an example of a ssDNA library preparation workflow. After nucleic acid extraction, ssDNA is processed by a ssDNA library preparation method using conversion-resistant splinted adapters, followed by EM conversion, amplification, target capture, and sequencing.

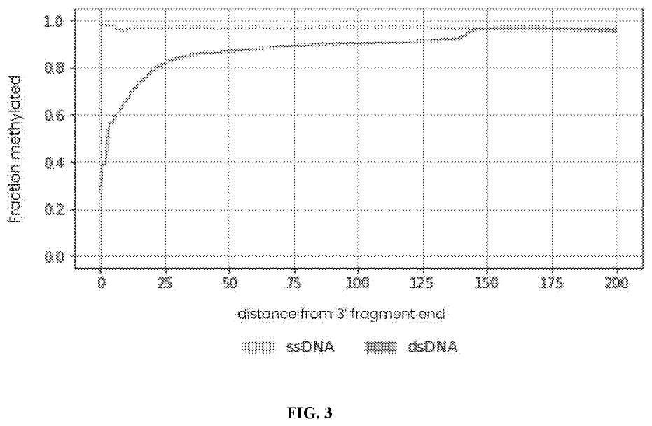

FIG. 3 provides a comparison of an example of a methylation signal relative to distance from the 3′ fragment ends using ssDNA library preparation versus dsDNA library preparation. When CpG methylation is measured in fragments that overlap hypermethylated genomic regions, fragments in dsDNA libraries exhibit a loss of methylation signal near the 3′ ends of fragments due to end repair (gray line (dsDNA)). In contrast, methylation signal is uniformly high across the length of fragments in ssDNA libraries (green line (ssDNA)).

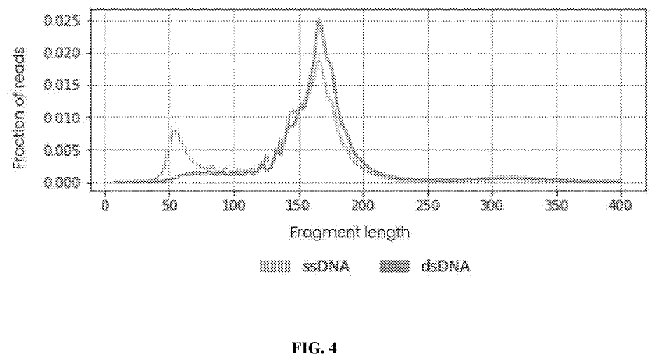

FIG. 4 provides a comparison of examples of captured reads relative to fragment length using ssDNA library preparation versus dsDNA library preparation. As illustrated, ssDNA library preparation methods are able to capture shorter cfDNA fragments corresponding to short ssDNA that are not captured by dsDNA library preparation methods.

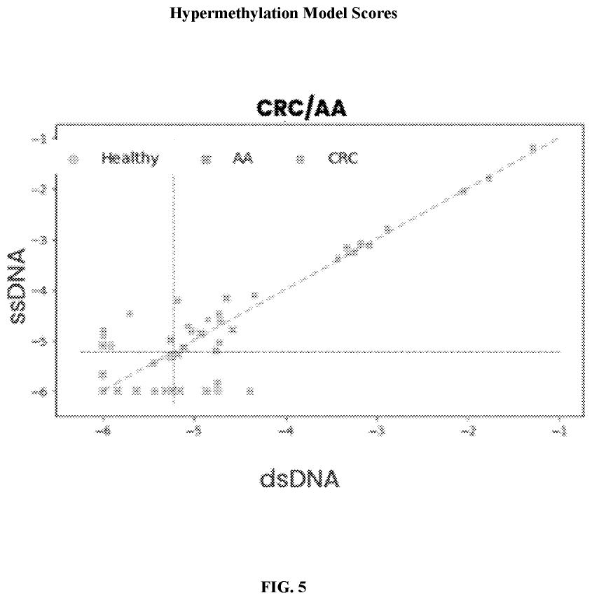

FIG. 5 provides a comparison of examples of hypermethylation model scores of cfDNA extracted from healthy/cancer-negative (NEG), advance adenoma (AA), and colorectal cancer (CRC) patient samples detected using ssDNA library preparation described herein versus other dsDNA library preparation methods. Hypermethylation rates of cfDNA derived from selected genomic regions distinguished AA and CRC patient-derived cfDNA from cancer-negative cfDNA when processed using both the ssDNA library preparation described herein and other dsDNA library preparation methods. This experiment demonstrates that the ssDNA library preparation method described herein yields equivalent methylation analysis (e.g., hypermethylation scores) as other dsDNA library preparation methods. This workflow is equivalent to end repair-dependent methods at discriminating cancer patient-derived from healthy patient-derived cfDNA samples, e.g., when using DNA hypermethylation analysis.

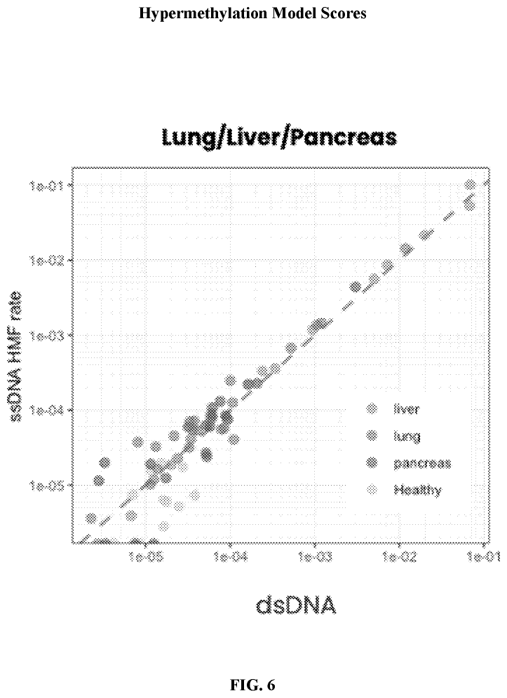

FIG. 6 provides a comparison of examples of hypermethylation model scores of cfDNA extracted from healthy/cancer-negative (NEG), liver cancer (Liver), lung cancer (Lung), and pancreatic cancer (Pancreas) patient samples detected using an ssDNA library preparation method as described herein versus other dsDNA library preparation methods. Hypermethylation rates of cfDNA derived from selected genomic regions distinguished liver cancer, lung cancer, and pancreatic cancer patient-derived cfDNA from cancer-negative cfDNA when processed using both the ssDNA library preparation described herein and other dsDNA library preparation methods. This experiment demonstrates that the ssDNA library preparation method described herein yield equivalent methylation analysis (e.g., hypermethylation scores) as other dsDNA library preparation methods. Thus, the ssDNA library preparation workflow described herein is equivalent to end repair-dependent methods at discriminating cancer patient-derived from healthy patient-derived cfDNA samples, e.g., when using DNA hypermethylation analysis.

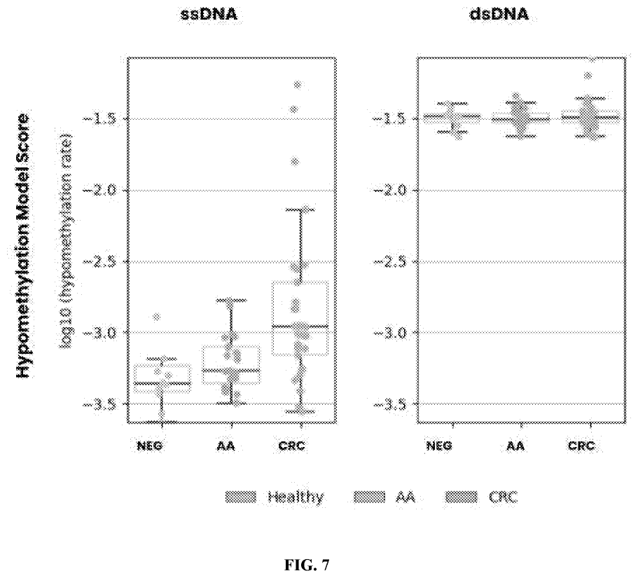

FIG. 7 provides a comparison of examples of hypomethylation scores of cfDNA extracted from healthy/cancer-negative (NEG), advance adenoma (AA), and colorectal cancer (CRC) patient samples detected using ssDNA library preparation described herein versus dsDNA library preparation. Hypomethylation rates of cfDNA derived from selected genomic regions distinguished AA and CRC patient-derived cfDNA from cancer-negative cfDNA when processed using ssDNA library preparation but not when processed using dsDNA library preparation. This experiment demonstrates that the ssDNA library preparation method described herein optimizes the recovery, methylation analysis, quantification, and target capture. This workflow can outperform end repair-dependent methods at discriminating cancer patient-derived from healthy patient-derived cfDNA samples, e.g., using DNA hypomethylation analysis.

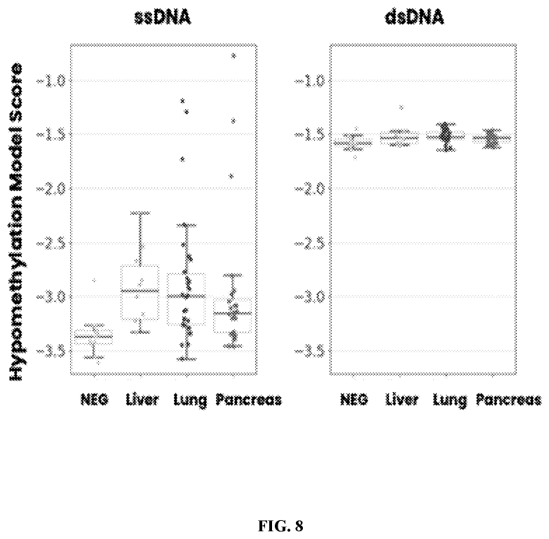

FIG. 8 provides a comparison of examples of hypomethylation scores of cfDNA extracted from healthy/cancer-negative (NEG), liver cancer (Liver), lung cancer (Lung), and pancreatic cancer (Pancreas) patient samples detected using an ssDNA library preparation method as described herein versus a dsDNA library preparation method. As shown in FIG. 8, hypomethylation rates of cfDNA derived from selected genomic regions distinguished liver cancer, lung cancer, and pancreatic cancer patient-derived cfDNA from cancer-negative cfDNA when processed using ssDNA library preparation but not when processed using dsDNA library preparation. This experiment provides additional evidence that the ssDNA library preparation method described herein optimizes the recovery, methylation analysis, quantification, and target capture across multiple cancer types which further provides that this workflow can outperform end repair-dependent methods at discriminating cancer patient-derived from healthy patient-derived cfDNA samples, e.g., using DNA hypomethylation analysis.

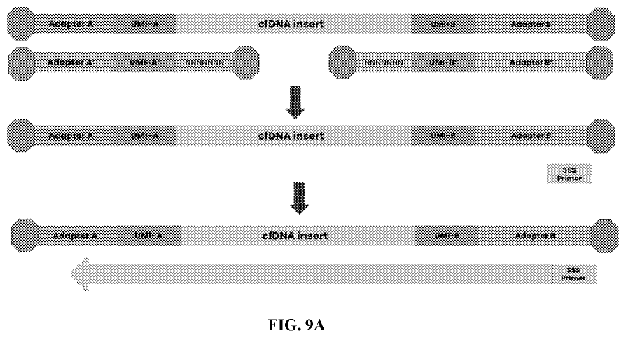

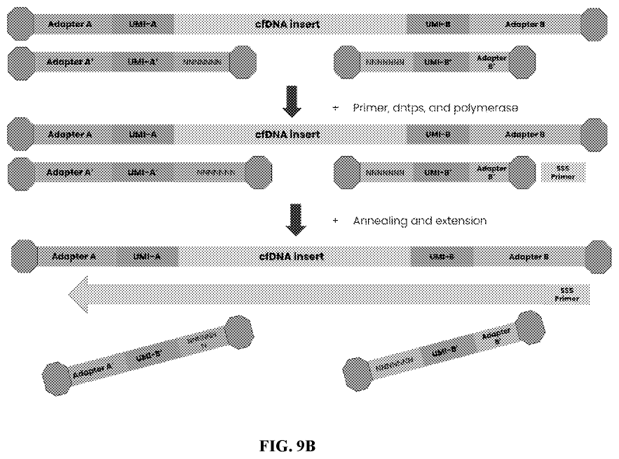

FIGS. 9A-9B illustrate methods by which ssDNA libraries may be converted into dsDNA libraries after ligation. ssDNA libraries may undergo a clean-up operation after ligation, after which they are added to a polymerization reaction containing reaction buffer, nucleotides, an oligonucleotide primer complementary to a portion of the 3′ adapter sequence, and a polymerase (FIG. 9A). Alternatively, ssDNA libraries may be generated using a splint adapter sequence so that they may be amplified without a clean-up operation to remove excess adapters (FIG. 9B).

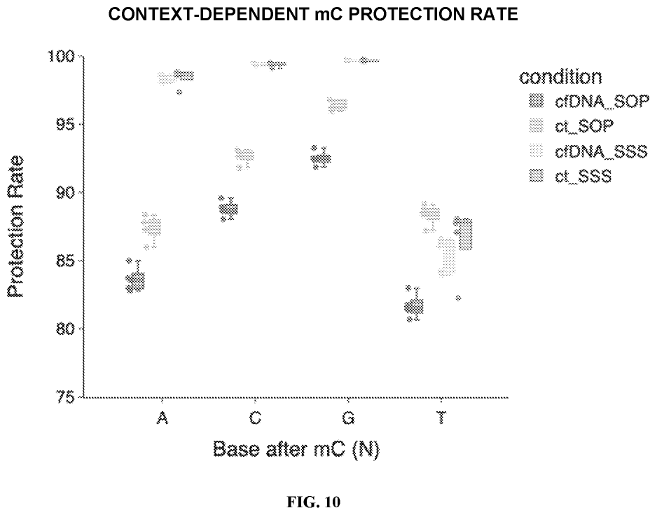

FIG. 10 provides a comparison of examples of the protection rate of methylated cytosines (mC) in ssDNA using a ssDNA library workflow with second strand synthesis (SSS) and with no second strand synthesis (SOP) after converting ssDNA libraries to dsDNA libraries. The protection rate varies depending on the identity of the base following the methylated cytosine (e.g., CpA, CpG, CpC, or CpT). cfDNA_SOP, cell-free DNA sample using ssDNA library workflow with no second strand synthesis; ct_SOP, contrived DNA sample using ssDNA library workflow with no second strand synthesis; cfDNA_SSS, cell-free DNA sample using ssDNA libraries with second strand synthesis; ct_SSS, contrived DNA sample using ssDNA libraries with second strand synthesis.

### Definitions

As used herein, singular terms, e.g., “a”, “an”, and “the” include both singular and plural referents unless the context clearly dictates otherwise.

As used herein, the term “plasma cell-free DNA”, “circulating free DNA”, “cell-free DNA”, or “cfDNA” generally refers to DNA molecules that circulate in the acellular portion of blood. Circulating nucleic acids in blood may arise from necrotic or apoptotic cells indicative of disease, such as cancer. In cancer, circulating DNA bears hallmark signs of the disease, including mutations in oncogenes and microsatellite alterations. These circulating DNA may be referred to as circulating tumor DNA (ctDNA). Viral genomic sequences, DNA, or RNA in plasma is a potential biomarker for disease.

In some embodiments, the cell-free fraction of blood is preferably blood serum or blood plasma. The term “cell-free fraction” of a biological sample, as used herein, generally refers to a fraction of the biological sample that is substantially free of cells. As used herein, the term “substantially free of cells” may refer to a preparation from the biological sample comprising fewer than about 20,000 cells per mL, fewer than about 2,000 cells per mL, fewer than about 200 cells per mL, or fewer than about 20 cells per mL. Genomic DNA (gDNA) refers to non-fragmented DNA that is released from white blood cells contaminating the blood cell-free fraction. To mitigate gDNA from contaminating samples, a highly controlled sample processing workflow may be implemented, and specimens may be screened against the presence of gDNA.

As used herein, the term “detect”, “detecting”, or “detection” of a status or outcome generally includes detecting the presence of an indication (such as cancer), detecting status or outcome, or detecting predisposition to a status or outcome.

As used herein, the term “diagnose” or “diagnosis” of a status or outcome generally includes predicting or diagnosing the status or outcome, determining predisposition to a status or outcome, monitoring treatment of patient, diagnosing a therapeutic response of a patient, prognosis of status or outcome, progression, and response to particular treatment.

As used herein, the term “location” generally refers to the position of a nucleotide in an identified strand in a nucleic acid molecule.

As used herein, the term “nucleic acid” generally refers to a DNA, RNA, DNA/RNA chimera or hybrid that may be single-strand (ss) or double-strand (ds). Nucleic acids may be genomic or derived from the genome of a eukaryotic or prokaryotic cell, or synthetic, cloned, amplified, or reverse transcribed. In certain embodiments of the methods and compositions, nucleic acid preferably refers to genomic DNA as the context requires.

As used herein, unless otherwise stated, the term “modified cytosine” generally refers to 5-methylcytosine (5mC), 5-hydroxymethylcytosine (5hmC), formyl modified cytosine, carboxy modified cytosine, 5-carboxylcytosine (5caC), or a cytosine modified by any other chemical group.

As used herein, the term “methylcytosine dioxygenase”, “dioxygenase”, or “oxygenase” generally refers to an enzyme that converts 5mC to 5hmC. Non-limiting examples of methylcytosine dioxygenases include, e.g., ten eleven translocation (TET) enzymes, e.g., TET1, TET2, TET3, Naegleria TET, and genetically engineered or non-genetically engineered variants thereof. TET2 is an example of a methylcytosine dioxygenase that oxidizes at least 90%, at least 92%, at least 94%, at least 96%, at least 98%, or at least 99% of all 5mC.

As used herein, the term “methylation conversion method” or “methylation enrichment method” or “methylation treatment method” generally refers to a method in which a nucleic acid molecule is subjected to conditions sufficient to convert unmethylated cytosines in the nucleic acid molecule to uracils. The method is useful for differentiating methylated cytosines from unmethylated cytosines in a nucleic acid molecule.

As used herein, the term “enzymatic methylation” or “enzymatic methyl” or “EM conversion” or “EM-seq” generally refers to a method in which a nucleic acid molecule is subjected to conditions sufficient to convert unmethylated cytosines in the nucleic acid molecule to uracils by treatment with one or more enzymes, e.g., a TET enzyme. In some cases, the method does not comprise treatment with bisulfite (e.g., chemical treatment).

As used herein, the term “conversion-resistant adapter”, “conversion-resistant primer”, “conversion-tolerant adapter”, or “conversion-tolerant primer” generally refers to nucleic acid molecules used as adapters or primers, respectively. These nucleic acid molecules are resistant to methylation conversion or alteration by a methylation enrichment method (e.g., a “deamination-resistant modified cytosine” or a methylation conversion agent resistant nucleotide).

As used herein, the terms “deamination-resistant modified cytosine” refers to one or more modified cytosine nucleotides in a conversion-resistant adapter that are not chemically or enzymatically altered by treatment with a methylation conversion agent to change the base pairing specificity of the nucleotide base. By way of example of a non-limiting example, propynyl-C and pyrrolo-C are deamination-resistant modified cytosines which are conversion resistant nucleotides and can be included in the conversion-resistant adapters used in the library preparation methods disclosed herein. These deamination-resistant modified cytosines within the conversion-resistant adapters are not deaminated when exposed to sodium bisulfite or methylation conversion enzymes (e.g., APOBEC-like enzymes).

As used herein, the term “methylation conversion agent resistant nucleotide” generally refers to a nucleotide comprising a nucleic acid base that is not chemically altered by treatment with a methylation conversion agent so as to change the base pairing specificity of the nucleotide base. Methylation conversion agent resistant nucleotides are capable of being incorporated by a nick translation enzyme in a primer extension reaction. For example, 5-methylcytosine (5mC) is a conversion-resistant nucleotide that may be used in conjunction with sodium bisulfite or enzymatic methylation conversion. Thus, 5-methylcytosine is not deaminated when exposed to sodium bisulfite or a methylation conversion enzyme. Instead of incorporating modified nucleotide bases to prevent base conversion, conversion-tolerant adapters or conversion-tolerant primers incorporate only unmodified bases to permit total base conversion during a conversion reaction for methylation sequencing. “Unmodified bases” in adapter/primer DNA sequences refer to conventional guanine, adenine, cytosine, and thymine.

As used herein, the term “cytidine deaminase” generally refers to an enzyme that deaminates cytosine (C) to form uracil (U). Non-limiting examples of cytidine deaminases include the apolipoprotein B mRNA-editing enzyme, catalytic polypeptide (APOBEC) family of cytidine deaminases, such as APOBEC3A. In any embodiment, a cytidine deaminase described herein may have an amino acid sequence that is at least 90% identical to (e.g., at least 95% identical to) the amino acid sequence of GenBank accession number AKE33285.1, which is the sequence of human APOBEC3A. In some embodiments, a cytidine deaminase described herein converts unmodified cytosine to uracil with an efficiency of at least 95%, at least 96%, at least 97%, at least 98%, or at least 99%, preferably at least 99%.

As used herein, the term “glucosyltransferase” or “GT” generally refers to an enzyme that catalyzes the transfer of a beta-D-glucosyl or alpha-D-glucosyl residue from UDP-glucose to 5hmC residue to form 5ghmC. APOBEC can convert 5hmC to U at a low rate relative to converting C or 5mC to U. An example of a GT is T4-betaGT (BGT). In one example, GT may be used concurrently with a dioxygenase. This combination ensures that deamination of 5hmC is blocked such that less than 5%, less than 3%, or less than 1% of 5hmC is converted to U by the deaminase. In another example, GT may be used together with dioxygenase in the same reaction mix with DNA such that the dioxygenase converts 5mC to 5hmC and 5caC, and the GT converts any residual 5hmC to 5ghmC to ensure only cytosine is deaminated.

As used herein, “a portion” of a nucleic acid sample and “an aliquot” of a nucleic acid sample generally are intended to have the same meaning and can be used interchangeably.

As used herein, the term “comparing” generally refers to analyzing two or more sequences relative to one another. In some cases, comparing may be performed by aligning two or more sequences with one another such that correspondingly positioned nucleotides are aligned with one another.

As used herein, the term “reference sequence” generally refers to the sequence of a fragment that is being analyzed. A reference sequence may be obtained from a public database or may be separately sequenced as part of an experiment. In some cases, the reference sequence may be hypothetical such that the reference sequence may be computationally deaminated (e.g., to change Cs into Us or Ts etc.) to allow a sequence comparison to be made.

As used herein, the terms “G”, “A”, “T”, “U”, “C”, “5mC”, “5fC”, “5caC”, “5hmC”, and “5ghmC” generally refer to nucleotides that contain guanidine (G), adenine (A), thymine (T), uracil (U), cytosine (C), 5-methylcytosine, 5-formylcytosine, 5-carboxylcytosine (5caC), 5-hydroxymethylcytosine, and 5-glucosylhydroxymethylcytosine, respectively. For clarity, each of C, 5fC, 5caC, 5mC, and 5ghmC is a different moiety.

As used herein, the term “minimal residual disease” or “MRD” generally refers to the small number of cancer cells in the body after cancer treatment. MRD testing may be performed to determine whether the cancer treatment is working and to guide further treatment plans. Various metrics can be used to assess MRD, including, but not limited to, response to treatment, tumor load, residual tumor post-surgery, relapse, secondary screen, primary screen, and cancer progression.

As used herein, the term “Next Generation Sequencing” or “NGS” generally applies to sequencing libraries of genomic fragments of a size of less than 1 kb.

As used herein, the term “healthy” or “normal” generally refers to a subject not having a disease, or a sample derived therefrom. While health is a dynamic state, the term may refer to the pathological state of a subject that lacks a referenced disease state, for example, cancer. In one example, when referring to a methylation profile that classifies subjects with cancer, the term “healthy” refers to an individual lacking cancer, such as CRC. While other diseases or states of health may be present in that subject, the term “healthy” may indicate the lack of a stated disease for comparison or classification purposes between subjects having and lacking a disease state, and samples derived therefrom.

As used herein, the term “threshold” generally refers to a value that is selected to discriminate, separate, or distinguish between two populations of subjects. In some embodiments, the threshold discriminates methylation status between a disease (e.g., malignant) state, and a non-disease (e.g., healthy) state. In some embodiments, the threshold discriminates between stages of disease (e.g., stage 1, stage 2, stage 3, or stage 4). Thresholds may be set according to the disease in question, and may be based on earlier analysis, e.g., of a training set or determined computationally on a set of inputs having a known characteristic (e.g., healthy, disease, or stage of disease). Thresholds may also be set for a gene region according to the predictive value of methylation at a particular site. Thresholds may be different for each methylation site, and data from multiple sites may be combined in the end analysis.

Although any methods and materials similar or equivalent to those described herein can also be used in the practice or testing of the present teachings, some example methods and materials are described herein.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

Cell-free DNA (cfDNA) sequencing may be a useful tool for cancer detection. DNA methylation analysis may be coupled with sequencing to determine whether a portion of cfDNA is likely to be pre-cancerous or tumor-derived. Many standard DNA library preparation methods use enzymatic end-repair processes to attach sequencing adapters. However, these processes can remove native methylation signals from DNA fragments, do not faithfully preserve molecule ends, and do not capture nicked or single-stranded DNA. These factors reduce the sensitivity of these methods for cancer detection (FIG. 1).

DNA methylation is a covalent modification of DNA and a stable inherited mark that can play an important role in repressing gene expression and regulating chromatin architecture. In humans, DNA methylation primarily occurs at cytosine residues in CpG dinucleotides. Unlike other dinucleotides, CpGs are not evenly distributed across the genome and can be concentrated in short CpG-rich DNA regions called CpG islands. In general, the majority of the CpG sites in the genome are ~70-75% methylated. However, methylation patterns differ from cell type to cell type, reflecting their role in regulating cell type-specific gene expression. In this manner, a cell's methylome can program the cell's terminal differentiation state to be, for instance, a neuron, a muscle cell, an immune cell, etc.

Further, various cell sub-types in a tissue can exhibit different methylation patterns. In cancer cells, CpG methylation can be deregulated, and aberrations in methylation patterns are some of the earliest events that occur in tumorigenesis. Methylation profiles in a given cancer type most closely resemble that of the tissue of origin of the cancer. Thus, aberrant methylation marks on a cfDNA fragment can be used to differentiate a cancer cell from a normal cell, and determine tissue type origin. In general, global CpG methylation levels decrease in cancer cells, but at specific loci, mean methylation levels (or % methylation) can vary at specific CpG sites in cancer cells relative to matched normal cells. Profiling differentially methylated CpGs (DMCs; single sites) or differentially methylated regions (DMRs; more than one site in a localized region) between normal and diseased cells allows identification of biomarkers of the disease. Such approach has led to development of the SEPT9 gene methylation assay (Epi proColon), which is the first FDA-approved blood-based diagnostic for colorectal cancer (CRC).

Methylation profiles, as used herein, can comprise both hypomethylation and hypermethylation of DNA analysis and/or either of them independently. Both hypomethylation and hypermethylation are relative terms and denote less (hypo) or more (hyper) methylation than in reference standard DNA. As applied specifically to cancer epigenetics, that reference standard can be DNA isolated from a normal tissue (e.g., non-cancerous).

Bisulfite conversion or bisulfite sequencing may be used for DNA methylation analysis. Bisulfite sequencing is a convenient and effective method of mapping DNA methylation to individual bases. Unfortunately, bisulfite conversion is a harsh and destructive process for cfDNA that leads to degradation of >90% of the sample DNA. Two main approaches to constructing bisulfite sequencing libraries are: (1) bisulfite conversion of DNA before library construction, which necessitates building ssDNA libraries; and (2) bisulfite conversion of DNA after dsDNA adapter ligation. Either case involves severe degradation of DNA, which can be problematic especially for cfDNA that is present at very low concentrations in plasma and is the limiting resource in liquid biopsy applications.

Alternatively, enzymatic methylation (EM) conversion may be used for DNA methylation analysis and sequencing. In one embodiment, methylation conversion is mediated by non-destructive enzymatic reactions, for example, using a ten eleven translocation (TET) enzyme and a cytosine-deaminating enzyme (e.g., the apolipoprotein B mRNA-editing enzyme, catalytic polypeptide (APOBEC) family of cytidine deaminases) to convert unmethylated (but not methylated) cytosines to uracils. Other embodiments, such as TET-assisted pyridine borane sequencing (TAPS), combine enzymatic reactions such as TET treatment together with chemical treatment (e.g., using pyridine borane).

The advent of next generation DNA sequencing offers advances in clinical medicine and basic research. However, while this technology has the capacity to generate hundreds of billions of nucleotides of DNA sequence in a single experiment, the error rate of approximately 1% results in hundreds of millions of sequencing mistakes. Such errors can be tolerated in some applications but become extremely problematic for “deep sequencing” of genetically heterogeneous mixtures, such as tumors or mixed microbial populations.

With existing methods, analyzing variants in cfDNA and methylation state in cfDNA requires two different sequencing assays and two different pools of cfDNA. This can be cost-prohibitive in terms of plasma/cfDNA input and associated costs. In addition, destruction of DNA by bisulfite can reduce the sensitivity of variant-calling methods that can work on bisulfite-converted DNA sequencing data (relative to enzymatic conversion). Thus, improved methods for analyzing methylation of cfDNA are needed to preserve the integrity of sample nucleic acid and enable improved accuracy of methylation state analysis at the whole genome or targeted level.

As used herein, the terms “including”, “includes”, “having”, “has”, “with”, or variants thereof are used in either the detailed description and/or the claims, such terms are intended to be construed as inclusive in a manner similar to the term “comprising”.

To the extent that ranges are used in the present disclosure, the ranges can be expressed herein as from “about” one particular value, and/or to “about” another particular value. When such a range is expressed in this manner, another embodiment includes from the one particular value and/or to the other particular value. In embodiments wherein the values are expressed as approximations, e.g., by use of the antecedent “about,” it will be understood that the specific value forms another embodiment. It will be further understood that the endpoints of each of the ranges are significant both in relation to the other endpoint, and independently of the other endpoint.

Several aspects of a ssDNA library preparation methods and systems of the present disclosure are described above with reference to example applications for illustration. It should be understood that numerous specific details, relationships, and methods are set forth to provide a full understanding of the disclosed methods and systems. A person having ordinary skill in the relevant art, however, will readily recognize that methods and systems can be practiced without one or more of the specific details or with other methods. The present disclosure is not limited by the particular ordering of acts or operations, as some acts may occur in different orders and/or simultaneously with other acts or operations. Furthermore, not all specified acts or operations are required to implement the method in accordance with the present disclosure.

### I. LIBRARY PREPARATION FOR ENZYMATIC METHYLATION SEQUENCING

In a first aspect, methods are provided for the preparation of a sequencing library. The methods described herein provide a ssDNA library that is acceptable for both next generation non-methylation and methylation sequencing applications, thereby providing sequencing data for two applications from a single sample. The resulting raw sequencing data may be used for methylation state analysis, as well as existing cfDNA analysis, such as copy number alterations, germline variant detection, somatic variant detection, nucleosome positioning, transcription factor profiling, chromatin immunoprecipitation, and the like.

### Adapter Ligation for Targeted Sequencing Applications

In one aspect, the present methods preserve the integrity and information of nucleic acid sequences for methylation profiling. In one example, combining ssDNA adapter ligation before enzymatic conversion preserves fragment endpoint information while increasing library complexity for target enrichment (or directly for genome-wide sequencing), thereby providing greater sensitivity to detect rare events, such as methylated ctDNA. The advantages of ssDNA adapter ligation and comparison of adapter ligation before methylation conversion is shown in FIG. 1.

In one example, nucleic acid adapters are ligated to the 5′ and 3′ ends of a population of nucleic acid fragments in a biological sample to produce a sequencing library. In another example, a collection of nucleic acid adapters is ligated to the nucleic acid fragments in a sample. The nucleic acid adapters described herein can optionally be conversion-tolerant nucleic acid adapters. In other examples, the nucleic acid adapters can optionally be conversion-resistant adapters which contain one or more deamination-resistant modified cytosines, including but not limited to, propynyl-C and pyrrolo-C. The collection of adapters can include equal parts of 4 base pair (bp), 5 bp, and 6 bp unique molecular identifier (UMI) sequences. The UMIs can be located adjacent to the library insert nucleic acid. During sequencing, the UMIs are also sequenced as a part of the read at the 5′ end. The collection of adapters can include single-length core UMIs, which can reduce sequencing complexity at the position corresponding to an invariant thymidine resulting in reduced sequencing quality. The first 4 bp of each UMI together can include a set of 4-bp (e.g., single length) core UMI sequences that have an edit distance of greater than or equal to 2, and are nucleotide and color balanced. Using single-length core UMIs in the presence of variable-length UMI sequences may facilitate the use of bioinformatic tools that are built for single-length UMIs for UMI extraction and de-duplication. Thus, the 4-bp core sequences may serve as a recognition sequence that informs the bioinformatic tool to trim 5, 6, or 7 bp sequences, thereby maintaining precise cfDNA end point information. A schematic illustrating the staggered adapters is shown in FIG. 2. The use of UMIs may permit read de-duplication as well as single-stranded error correction. In another example, unique dual indexes (UDI) are additional sequences that may be added to the UMI-containing adapters during library preparation to provide sample barcoding and de-multiplexing of samples after sequencing. In various examples, the UDI sequences are more than or equal to 4 bp, 5 bp, 6 bp, 7 bp, 8 bp, or 12 bp in length. In various examples, the UDI sequences are less than or equal to 12 bp, 8 bp, 7 bp, 6 bp, 5 bp, or 4 bp in length.

In various embodiments, the nucleic acid adapters may include UMIs of 4 bp to 6 bp in length. The UMIs can be designed to be non-unique (e.g., drawn from a specific, constrained set of sequences).

In one embodiment, some UMIs contain one or more methylcytosine bases. The efficiency of the enzymatic methylation conversion reactions (including TET oxidation and APOBEC deamination) can be assessed based on the fraction of UMIs that do not match the specific, constrained set of designed UMI sequences by a UMI mismatch rate. The UMI mismatch rate may be used as an embedded quality control metric to assess sequencing library quality. In addition, if perfect UMI matches are required in the bioinformatics pipeline, then the UMI mismatch rate may be used as a filter to remove individual reads that may be of lower quality due to incomplete conversion.

In various embodiments, the UMI mismatch rate is less than or equal to 6%, less than or equal to 5%, less than or equal to 4%, less than or equal to 3%, or less than or equal to 2%.

In another embodiment, the UMIs contain one or more cytosines containing modifications that may be used to monitor the enzymatic activities. Non-limiting examples of these modified bases include 5-methylcytosine, 5-hydroxymethylcytosine, 5-formylcytosine, and 5-carboxylcytosine.

The efficiency of subsequent processes such as DNA purification or enzymatic methyl conversion may be improved by converting ssDNA libraries to dsDNA libraries. In some embodiments, ssDNA to dsDNA conversion may be performed using a DNA polymerase and a primer complementary to the 3′ end of the adapter sequence. The primer anneals to the adapter-ligated nucleic acid at the 3′ end of the adapter sequence to initiate an extension reaction in the presence of polymerase and deoxynucleotide triphosphates (dNTPs). In some embodiments, ssDNA to dsDNA conversion may occur after a purification (“clean up”) operation between ligation and DNA polymerization to remove free, excess adapters and exchange reaction buffers. In other embodiments, ssDNA to dsDNA conversion reaction components (including dNTPs, polymerase, and primer) are added directly to the ligation mixture without a purification operation. In some embodiments, the splint component of the 3′ adapter is truncated such that the splint component incompletely overlaps with the primer annealing site for the primer extension. In this way, a primer complementary to the 3′ end of the adapter sequence can be annealed at the primer annealing site. Second strand synthesis (SSS) primers may or may not have terminal modifiers on the 3′ end, 5′ end, or both.

### II. TARGETED METHYLATION SEQUENCING

In targeted methylation sequencing approaches, targeted regions in a biological sample, such as cfDNA, are analyzed to determine the methylation state of the target gene sequences. In some embodiments, the target region comprises, or hybridizes under stringent conditions to, contiguous nucleotides of target regions of interest, such as at least about 16 contiguous nucleotides of a target region of interest. In different examples, targeted sequencing may be accomplished using hybridization capture and amplicon sequencing approaches.

### A. Hybridization Capture

The hybridization method provided herein may be used in various formats of nucleic acid hybridizations, such as in-solution hybridization and such as hybridization on a solid support (e.g., Northern, Southern, and in situ hybridization on membranes, microarrays, and cell/tissue slides). In particular, the method is suitable for in-solution hybrid capture for target enrichment of certain types of genomic DNA sequences (e.g., exons) employed in targeted next-generation sequencing. For hybrid capture approaches, a cell-free nucleic acid sample is subjected to library preparation. As used herein, “library preparation” comprises adapter ligation of ssDNA as described herein, or any other preparation performed on the cell-free DNA to permit subsequent sequencing of DNA. In certain examples, a prepared cell-free nucleic acid library sequence contains adapters, sequence tags, and index barcodes that are ligated onto cell-free nucleic acid sample molecules. Various commercially available kits are available to facilitate library preparation for next-generation sequencing approaches. Next-generation sequencing library construction may comprise preparing nucleic acids targets using a coordinated series of enzymatic reactions to produce a random collection of DNA fragments, of specific size, for high throughput sequencing. Advances and the development of various library preparation technologies have expanded the application of next-generation sequencing to fields such as transcriptomics and epigenetics.

Improvements in sequencing technologies have resulted in changes and improvements to library preparation. Next-generation sequencing library preparation kits used herein include those developed by companies such as Agilent, Bioo Scientific, Claret Bioscience, Kapa Biosystems, New England Biolabs, Illumina, Life Technologies, Pacific Biosciences, and Roche.

In various examples for targeted capture gene panels, various library preparation kits may be selected from Nextera Flex (Illumina), IonAmpliseq (Thermo Fisher Scientific), and Genexus (Thermo Fisher Scientific), Agilent ClearSeq (Illumina), Agilent SureSelect Capture (Illumina), Archer FusionPlex (Illumina), BiooScientific NEXTflex (Illumina), IDT xGen (Illumina), Illumina TruSight (Illumina), Nimblegene SeqCap (Illumina), and Qiagen GeneRead (Illumina).

In some embodiments, the hybrid capture method is carried out on the prepared library sequences using specific probes. As used herein, the term “specific probe” may refer to a probe that is specific for a known methylation site. In some embodiments, the specific probes are designed based on using the human genome as a reference sequence and using specified genomic regions known to have methylation sites as target sequences. Specifically, the genomic region known to have methylation sites may comprise at least one of the following: a promoter region, a CpG island region, a CGI shore region, and an imprinted gene region. Therefore, when carrying out the hybrid capture by using the specific probes of some embodiments, the sequences in the sample genome that are complementary to the target sequences, e.g., regions in the sample genome known to have methylation sites (which are also referred to as “specified genomic regions” herein), may be captured efficiently.

By way of example, the methylated regions described herein may be used for designing the specific probes. In some embodiments, the specific probes are designed using commercially available methods, such as, for example, an eArray system. The length of the probes may be sufficient to hybridize with sufficient specificity to the methylated region of interest. In various examples, the probe is a 10-mer, 11-mer, 12-mer, 13-mer, 14-mer 15-mer, 16-mer, 17-mer, 18-mer, 19-mer, or 20-mer. In other embodiments, the probes are 50-200 nucleotides in length. In certain embodiments, the probes are 80-150 nucleotides in length. In still other embodiments, the probes are 100-130 nucleotides in length. In some embodiments, the probes are more than or equal to 50, 60, 70, 80, 90, 100, 110, 120, 130, 140, 150, 160, 170, 180, 190, or 200 nucleotides in length. In some embodiments, the probes are less than or equal to 200, 190, 180, 170, 160, 150, 140, 130, 120, 110, 100, 90, 80, 70, 60, or 50 nucleotides in length.

Targeted regions for methylation analysis may be screened out by making use of database resources (such as gene ontology). According to the principle of complementary base pairing, a single-stranded capture probe may be combined with a single-stranded target sequence complementarily, so as to capture the target region successfully. In some embodiments, the designed probes may be designed as a solid capture chip (wherein the probes are immobilized on a solid support) or as a liquid capture chip (wherein the probes are free in the liquid). However, due to limiting factors, such as probe length, probe density, and high cost, the solid capture chip is rarely used, whereas the liquid capture chip is used more frequently.

In some embodiments, compared with normal sequences (where the average content of A, T, C, and G base is each 25%), GC-rich sequences (where the average content of GC bases is higher than 60%) may lead to the reduction of capture efficiency because of the molecular structures of C and G bases. For the key research regions, for example, CGI regions (CpG islands), an increased amount of the probes may be required to obtain sufficient and accurate CGI data.

### B. Amplicon-Based Sequencing

Fragments of the converted ssDNA may be amplified. In some embodiments, the amplifying is carried out with primers designed to anneal to methylation converted target sequences having at least one methylated site therein. Methylation sequencing conversion results in unmethylated cytosines being converted to uracil, while 5-methylcytosine is unaffected. “Converted target sequences” may refer to sequences in which cytosines known to be methylation sites are fixed as “C” (cytosine), whereas cytosines known to be unmethylated are fixed as “U” (uracil); which may be treated as “T” (thymine) for primer design purposes.

In various examples, the source of the DNA is cell-free DNA obtained from whole blood, plasma, serum, or genomic DNA extracted from cells or tissue. In some embodiments, the size of the amplified fragment is between about 100 and 200 base pairs in length. In some embodiments, the DNA source is extracted from cellular sources (e.g., tissues, biopsies, cell lines), and the amplified fragment is between about 100 and 350 base pairs in length. In some embodiments, the amplified fragment comprises at least one 20 base pair sequence comprising at least one, at least two, at least three, or more than three CpG dinucleotides. The amplification may be carried out using sets of primer oligonucleotides according to the present disclosure, and may use a heat-stable polymerase. The amplification of several DNA segments may be carried out simultaneously in one and the same reaction vessel. In some embodiments, two or more fragments are amplified simultaneously. For example, the amplification may be carried out using a polymerase chain reaction (PCR).

Primers designed to target such sequences may exhibit a degree of bias towards converted methylated sequences. In some embodiments, the PCR primers are designed to be methylation specific for targeted methylation-sequencing applications. Methylation specific primers may allow for greater sensitivity in some applications. For instance, primers may be designed to include a discriminatory nucleotide (specific to a methylated sequence following bisulfite conversion) that is positioned to achieve optimal discrimination, e.g., in PCR applications. The discriminatory may be positioned at the 3′ ultimate or penultimate position.

In some embodiments, the primers are designed to amplify DNA fragments 75 to 350 base pair (bp) in length, which is the general size range for circulating DNA. Optimizing primer design to account for a target size may increase sensitivity of a method described herein. The primers may be designed to amplify regions that are about 50 to 200, about 75 to 150, or about 100 or 125 bp in length.

In one embodiment, the amplification operation comprises using primers that contain a unique dual index (UDI) sequence.

In one embodiment, the UDI sequences are more than or equal to 4 base pairs (bp), 5 bp, 6 bp, 7 bp, 8 bp, 9 bp, 10 bp, 11 bp, or 12 bp in length. In one embodiment, the UDI sequences are less than or equal to 12 bp, 11 bp, 10 bp, 9 bp, 8 bp, 7 bp, 6 bp, 5 bp, or 4 bp in length.

In some embodiments, the methylation status of pre-selected CpG positions within the nucleic acid sequences is detected by the amplicon-based approach using of methylation-specific PCR (MSP) primer oligonucleotides. The use of methylation-specific primers for the amplification of converted methylated ssDNA allows the differentiation between methylated and unmethylated nucleic acids. MSP primers pairs contain at least one primer which hybridizes to a converted CpG dinucleotide. Therefore, the sequence of said primers comprises at least one CpG, TpG, or CpA dinucleotide. MSP primers that are specific for non-methylated DNA contain a “T” at the 3′ position of the C position in the CpG. Therefore, the base sequence of these primers may include a sequence having a length of at least 18 nucleotides which hybridizes to a pretreated nucleic acid sequence and sequences complementary thereto, and the base sequence has at least one CpG, TpG, or CpA dinucleotide. In some embodiments of the method, the MSP primers have between 2 and 5 CpG, TpG, or CpA dinucleotides. In some embodiments, the dinucleotides are located within the 3′ half of the primer, e.g., for a primer having 18 bases in length, the specified dinucleotides are located within the first 9 bases from the 3′ end of the sequence. In addition to the CpG, TpG, or CpA dinucleotides, the primers may further include several methyl converted bases (e.g., cytosine converted to thymine, or on the hybridizing strand, guanine converted to adenosine). In some embodiments, the primers are designed to have no more than 2 cytosine and/or guanine bases.

In some embodiments, each of the regions is amplified in sections using multiple primer pairs. In some embodiments, these sections are non-overlapping. The sections may be immediately adjacent or spaced apart (e.g., spaced apart up to 10 base pairs (bp), 20 bp, 30 bp, 40 bp, or 50 bp). Since target regions (including CpG islands, CpG shores, and/or CpG shelves) are usually longer than 75 to 150 bp, this example permits the methylation status of sites across more (or all) of a given target region to be assessed.

Primers may be designed for target regions using suitable tools such as Primer3, Primer3Plus, Primer-BLAST, etc. As discussed, methylation enrichment conversion results in unmethylated cytosine converting to uracil and methylated cytosine converting to thymine. Thus, primer positioning or targeting may make use of converted sequences, depending on the degree of methylation specificity required.

### C. Enzymatic Conversion for DNA Methylation Sequencing Applications

Bisulfite conversion may be damaging to input DNA and may result in overall yield loss, fragmentation, and biased sequencing data. As an alternative, enzymatic methyl conversion can be used in methylation sequencing workflows. Examples of enzymatic methyl conversion workflows include enzymatic methyl-seq (EM-seq) and TET-assisted pyridine borane sequencing (TAPS).

EM-seq is a minimally destructive conversion methylation sequencing method for converting cytosines to uracils in nucleic acid. This bisulfite-free method preserves the length of nucleic acid molecules while achieving conversion rates similar to bisulfite sequencing. Further, EM-Seq can result in higher sequencing quality scores for cytosine and guanine base pairs, and can provide a more even coverage of various genomic features, such as CpG islands. EM-Seq comprises two sets of enzymatic reactions. In the initial reaction, a ten eleven translocation (TET) enzyme (e.g., TET1, TET2, TET3, Naegleria TET, and genetically engineered versions and/or variants thereof) and a β-glucosyltransferase (e.g., T4 BGT) convert 5mC and 5hmC into products that cannot be deaminated, or are resistant to deamination, by a cytosine-deaminating enzyme (e.g., APOBEC). In the second reaction, a cytosine-deaminating enzyme (e.g., APOBEC) deaminates unmodified (e.g., unmethylated) cytosines by converting them to uracils.

In another embodiment, TAPS can be used in enzymatic methylation sequencing workflows. TAPS is a minimally-destructive conversion methylation sequencing method for converting cytosines to uracil in nucleic acid. This bisulfite-free method allows minimal degradation of DNA, and thus preserves the length of nucleic acid molecules while achieving conversion rates similar to sodium bisulfite sequencing. TAPS can result in higher sequencing quality scores for cytosines and guanine base pairs, and can provide a more even coverage of various genomic features, such as CpG islands.

In TAPS, a ten eleven translocation enzyme (e.g., TET1) is used to oxidize both 5mC and 5hmC to 5caC. Pyridine borane is used to reduce 5caC to dihydrouracil, a uracil derivative that is then converted to thymine after PCR. TAPS can be performed in two other ways: TAPSß and chemical-assisted pyridine borane sequencing (CAPS). In TAPSB, β-glucosyltransferase is used to label 5hmC with glucose to protect 5hmC from the oxidation and reduction reactions and allows for specific detection of 5mC. In CAPS, potassium perruthenate acts as the chemical replacement for Tet1 and specifically oxidizes 5hmC, thus allowing for direct detection.

In one example of enzymatic methyl conversion, the combination of enzymatic conversion of unmodified C to U, and staggering UMI adapters in line with the library insert, are useful for targeted sequencing of methylation libraries. For low-depth sequencing applications, this combination may permit reduced volume inputs of plasma or mass inputs of cfDNA as compared to bisulfite conversion sequencing because sample cfDNA is not degraded to the same extent.

For high-depth sequencing applications, higher depth sequencing may be obtained as compared to bisulfite conversion sequencing from similar inputs of plasma or cfDNA because cfDNA is not degraded to the same extent.

In one example, the cytosines present in adapter nucleic acids are modified with a 5-methyl group or 5-hydroxymethyl group to prevent C-to-T conversion in the adapters.

One advantage of this approach is that adapter ligation before conversion maintains fragment endpoint and length information as compared to an approach that performs bisulfite conversion followed by ssDNA adapter ligation. The considerable degradation of nucleic acid before ligating adapters may result in loss of informative fragment endpoint and length information.

Enzymatic unmodified C conversion to U is less harsh on sample nucleic acid fragments and may result in more complete and uniform coverage as compared to bisulfite conversion methods. Bisulfite degradation of DNA is not uniform such that some sequences are preferentially degraded over others, including CG dinucleotides, which are the very sites being interrogated in methylation sequencing. Thus, the enzymatic approach provides a higher coverage of CpG sites than bisulfite conversion methods using the same number of unique reads, and greater uniformity of captured reads in target enrichment applications. Furthermore, non-bisulfite methods (e.g., enzymatic and TAPS-like chemical conversion) provide increased resolution of biological signal, and specifically, the ability to differentiate 5mC and 5hmC methylation in a nucleic acid sequence. This information and additional resolution may be informative in computational approaches and other methods.

In some examples, subjecting the DNA or the barcoded DNA to enzymatic reactions that convert cytosine nucleobases of the DNA or the barcoded DNA into uracil nucleobases includes “performing enzymatic conversion”.

In various examples, glucosylation and oxidation reactions overcome the observed inherent deamination of 5hmC and 5mC by deaminases. Deaminases converts 5mC and unmodified C to U, but does not convert 5ghmC and 5caC. Non-limiting examples of deaminases include APOBEC (apolipoprotein B mRNA editing enzyme, catalytic polypeptide-like). Embodiments described herein utilize enzymes that substantially have no sequence bias in glucosylation, oxidation, and deamination of cytosine. Moreover, these embodiments provide substantially no non-specific damage of the DNA during the glucosylation, oxidation, and deamination reactions.

In some embodiments, a glucosyltransferase (GT), e.g., beta-glucosyltransferase (BGT), is utilized to covalently link glucose to 5hmC to protect this modified base from deamination. Other enzymatic or chemical reactions may be used for modifying the 5hmC to achieve the same effect.

In general, and in one aspect, a method provided herein includes (a) treating an aliquot (portion) of a nucleic acid sample with a dioxygenase, e.g., TET2, and BGT in a reaction mix to produce a reaction product in which substantially all modified cytosines (Cs) are either oxidized, or in the case of 5hmC, glucosylated; and (b) treating this reaction product with cytidine deaminase to convert substantially all unmodified Cs to U. The term “modified” cytosines used in throughout these examples and embodiments refers to one or more of 5mC, 5hmC, 5ghmC, 5fC, and 5caC where oxidation to completion of 5mC, 5hmC, and 5fC results in 5caC. BGT reacts with 5hmC only. However, some of the 5hmC may be converted to 5fC and then to 5caC by the dioxygenase before glucosylation occurs. In the presence of the dioxygenase, 5mC is largely oxidized to completion to 5caC, but some residual 5hmC may be produced. However, residual 5hmC may be glucosylated by BGT to prevent the low deamination rate of 5hmC that may otherwise reduce accuracy of methylation sequencing.

The method described therefore largely discriminates between unmodified and modified cytosine by treating the nucleic acid with a dioxygenase before deamination. However, the amount of naturally occurring 5mC in genomic DNA may substantially exceed the amount of 5hmC, which in turn, may exceed the amount of naturally occurring 5fC and 5caC. Hence, the amount of naturally occurring modified cytosine generally is considered to be an approximate of the amount of naturally occurring 5mC.

In one example, the method can be adapted to perform 5hmC sequencing. The 5hmC sequencing method may further include: treating an aliquot of the nucleic acid sample with BGT in the absence of dioxygenase, followed by treatment with cytidine deaminase to produce a reaction product in which substantially all the 5hmCs in the aliquot are glucosylated, and substantially all the unmodified Cs and 5mCs are converted to Us. After PCR amplification, the Us are converted to Ts, and thus, cytosine and 5mC become indistinguishable when sequenced. The resultant reaction product can be sequenced and compared to a reference sequence to differentiate 5hmCs from Cs and from 5mCs. Differentiation of these moieties allows mapping of these modified nucleotides to a reference sequence, for example, a reference sequence from a database or an independently determined reference sequence.

In some embodiments, the dioxygenase with BGT plus deaminase reaction product or an amplification product thereof may be sequenced to determine which Cs are methylated (which may include a minor fraction of 5hmC) and which Cs are unmodified. In some embodiments, the βGT without dioxygenase plus deaminase reaction product or an amplification product thereof may be sequenced to determine which Cs are hydroxymethylated and which Cs are not hydroxymethylated. In some embodiments, the βGT without dioxygenase plus deaminase reaction product or an amplification product thereof may be sequenced to determine which Cs are hydroxymethylated and which Cs are unmodified. A reference DNA may be generated by sequencing a resulting reaction product that is produced by not reacting the nucleic acid sample with any one of dioxygenase, βGT, and deaminase. Alternatively, a reference sequence is a known reference sequence, e.g., from a database of sequences.

In one embodiment, the sequence of the dioxygenase with βGT plus deaminase reaction product can be compared to the reference sequence. Optionally, this can also be compared to the sequence of the βGT (without dioxygenase) plus deaminase reaction product to determine which cytosines in the nucleic acid sample are modified by a methyl versus a hydroxymethyl group.

In one aspect, a method is provided for performing targeted methylation sequencing of a cell-free DNA (cfDNA) sample from a subject, comprising:
a) ligating a conversion-tolerant nucleic acid adapter to a single-stranded nucleic acid molecule of the cfDNA sample, wherein the single-stranded nucleic acid molecule comprises unconverted nucleic acids;
b) enzymatically converting unmethylated cytosines to uracils in the single-stranded nucleic acid molecule to produce converted nucleic acids;
c) amplifying the converted nucleic acids by polymerase chain reaction;
d) probing the converted nucleic acids with nucleic acid probes that are complementary to a pre-identified panel of CpG or CH loci to enrich for sequences corresponding to the pre-identified panel of CpG or CH loci;
e) determining the nucleic acid sequence of the converted nucleic acids at a depth of >100×; and
f) comparing the nucleic acid sequence of the converted nucleic acids to a reference nucleic acid sequence of the pre-identified panel of CpG or CH loci to determine the methylation profile of the cfDNA sample from the subject.

In some embodiments, the conversion-tolerant nucleic acid adapter is a conversion-resistant nucleic acid adapter comprising one or more deamination-resistant modified cytosines including, but not limited to, propynyl-C and pyrrolo-C. In one embodiment, the conversion-resistant adaptors can comprise one or more propynyl-C residues. In another embodiment, the conversion-resistant adaptors can comprise one or more pyrrolo-C residues. In still another embodiment, the conversion-resistant adaptors can comprise a combination of propynyl-C and pyrrolo-C residues. If the test converted nucleic acid sequence is a T that corresponds to the reference C at a specified CpG locus, then the C was unmethylated in the original test nucleic acid fragment. In contrast, if the test converted nucleic acid sequence and the reference sequence are both C at a specified CpG locus, then the C was methylated in the original test nucleic acid fragment.

In one example, the nucleic acid sequence of the converted nucleic acid molecules is sequenced at a depth of between about 50-500×, about 25-1000×, about 50-500×, about 250-750×, about 500-200×, about 750-1500×, or about 100-2000×. In some embodiments, a nucleic acid sequence is sequenced at a depth of >100× or >500×.

In one example, the nucleic acid sequence of the converted nucleic acid molecules is sequenced at a depth of about 500×, about 1000×, about 2000×, about 3000×, about 4000×, about 5000×, about 6000×, about 7000×, about 8000×, about 9000×, about 10000×, or greater than 5000×.

In one example, the nucleic acid sequence of the converted nucleic acid molecules is sequenced at a depth of about 300× unique, about 400× unique, about 500× unique, about 600× unique, about 700× unique, about 800× unique, about 900× unique, or about 1000× unique, or greater than 500× unique.

### D. Target Enrichment Sequencing Applications

Further provided are methods for enriching methylated regions of interest in target capture applications during sequencing. A potential problem with applying target enrichment capture panels with DNA methylation libraries is a low rate of on-target reads/high rate of off-target DNA fragment capture. For every region in a panel, probes may be designed to target DNA derived from methylated CpGs or DNA derived from unmethylated CpGs. In either probe type, every CpG site along the region is considered unmethylated or methylated, as appropriate for the probe type. The probes may be hybridized to library molecules after bisulfite/enzymatic conversion and PCR amplification. Only the library molecules that are captured by the probes are then sequenced. This method has the advantage of reducing sequencing costs since only a small fraction of the genome is sequenced. In one example, about 0.1% of the genome is sequenced. In one example, about 0.3% of the genome is sequenced. In one example, about 0.5% of the genome is sequenced. In one example, about 0.7% of the genome is sequenced. In one example, about 1% of the genome is sequenced. In other examples, more than or equal to about 2%, about 3%, about 4%, about 5%, about 6%, about 7%, about 8%, about 9%, or about 10% of the genome is sequenced. In other examples, less than or equal to about 10%, about 9%, about 8%, about 7%, about 6%, about 5%, about 4%, about 3%, or about 2% of the genome is sequenced.

Significant off-target capture rates may occur with target capture enrichment approaches on both bisulfite and enzymatic converted libraries. Off-target capture rates are partly due to C-to-T conversion of all cytosines that are not in CpG sites in both types of probes that are hybridize to DNA derived from methylated CpGs. Decreasing cytosine content in probes leads to reduced sequence complexity, and hence, less specificity of probes hybridizing to target library molecules.

As used herein, the terms “methylated probes” and “unmethylated probes” refer to probes that are used to hybridize to methylated and unmethylated CpGs, respectively, in a post-conversion nucleic acid sequence. Probes may be designed to recognize post-conversion nucleic acids sequences. In post-conversion methylated CpG probes, Cs remain as Cs after conversion. In post-conversion unmethylated CpG probes, Cs are converted to Ts after conversion. In both post-conversion methylated and unmethylated probes, all Cs in a non-CpG dinucleotide are converted to Ts after conversion.

Methylated probes retain some cytosines (e.g., cytosines in CpG sites). In contrast, all cytosines are converted to thymines in unmethylated probes. Unmethylated probes are less complex than methylated probes and may likely preferentially contribute to off-target capture rates. In one example, probes that hybridize to DNA derived from methylated CpGs are used for target enrichment methods. In one example, probes having a substantially complementary sequence to a target that hybridize to DNA derived from methylated CpGs are used for target enrichment methods.

Probes that hybridize to DNA derived from methylated CpGs for target enrichment can be chosen to accomplish different aspects. Target capture hybridization reactions occur at a single temperature. However, the optimal melting temperature (Tm) of probes that hybridize to DNA derived from methylated CpGs is, on average, higher than the Tm of probes that are not designed to hybridize to DNA derived from methylated CpGs.

Cytosines base pairing involves three hydrogen bonds, whereas thymines base pairing involves two hydrogen bonds. Conversion of cytosines to thymines in probes lowers the Tm of the probe due to a decrease in hydrogen bonding. Probes designed to collect DNA derived from methylated fragments may contain more cytosines than the set of probes designed to collect the unmethylated fragments corresponding to the same genomic regions. As a result, methylated-fragment-targeting probes may have an elevated Tm relative to corresponding unmethylated probes. As the number of CpG sites increases in a region, the difference in melting temperatures between methylated and unmethylated probes also increases. Probes with higher melting temperatures may hybridize to a target DNA fragment more efficiently than probes with lower melting temperatures. Hybridization temperatures are generally selected to be relatively high to promote on-target capture. However, at many hybridization temperatures, methylated probes may more efficiently hybridized than unmethylated probes because of higher melting temperatures resulting from retention of some cytosines. Higher melting temperatures may lead to a bias toward higher % of CpG methylation levels measured by target capture hybridization approaches as compared to levels measured by sequencing of pre-capture libraries.

In one example, only a single probe type, methylated or unmethylated, is used in a hybridization reaction to enrich for hypermethylated or hypomethylated library molecules, respectively. Using a single type of methylated or unmethylated probe can circumvent the problem of divergent melting temperatures between the probe types. Using a single probe type may also promote more efficient capture (or enrichment) of the same DNA fragment type. In one example, the use of only methylated probes provides preferential binding of hypermethylated over hypomethylated regions of interest (ROIs). In another example, the use of only unmethylated probes provides enrichment of unmethylated ROIs.

Using only a single probe type also allows higher hybridization temperatures to be used to decrease off-target capture without affecting the relative balance of methylated to unmethylated ROI capture. Thus, probe panels can be designed based on the desire to enrich for hypermethylated or hypomethylated DNA fragments. In one example, where quantitation of both hypermethylated and hypomethylated DNA fragments is desired, two parallel, but separate, hybridization reactions are employed for both methylation states.

### E. Methylation Analysis

In various examples, enzymatic methylation sequencing results are used to analyze the methylation state of nucleic acids in a biological sample. In one example, whole genome enzymatic methyl sequencing (“WG EM-seq”) provides high resolution sequencing by characterizing DNA methylation of nearly every cytidine nucleotide in the genome. Other targeted methods, such as targeted enzymatic methyl sequencing (“TEM-seq”), may be useful for methylation analysis.

In other examples, assays that may be used for bisulfite conversion can be employed for minimally-destructive conversion methods, such as enzymatic conversion, TAPS, and CAPS. In various examples, assays used for methylation analysis may be mass spectrometry, methylation-specific PCR (MSP), reduced representation bisulfite sequencing (RRBS), HELP assay, GLAD-PCR assay, ChIP-on-chip assays, restriction landmark genomic scanning, methylated DNA immunoprecipitation (MeDIP), pyrosequencing of bisulfite treated DNA, molecular break light assay, methyl sensitive Southern Blotting, High Resolution Melt Analysis (HRM or HRMA), ancient DNA methylation reconstruction, or Methylation Sensitive Single Nucleotide Primer Extension Assay (msSNuPE).

The methylation profile of cfDNA can be identified by applying sequence alignment methods to map methyl-seq reads from whole genome or targeted methyl sequencing of a human reference genome. Non-limiting examples of sequence alignment methods include bwa-meth, bismark, Last, GSNAP, BSMAP, NovoAlign, Bison, Metagenomic Phylogenetic Analysis (for example, MetaPhlAn2), BLAT, Burrows-Wheeler Aligner (BWA), Bowtie, Bowtie2, Bfast, BioScope, CLC bio, Cloudburst, Eland/Eland2, GenomeMapper, GnuMap, Karma, MAQ, MOM, Mosaik, MrFAST/MrsFAST, PASS, PerM, RazerS, RMAP, SSAHA2, Segemehl, SeqMap, SHRIMP, Slider/SliderII, Srprism, Stampy, vmatch, ZOOM, and the SOAP/SOAP2 alignment tool.

### F. Identifying Somatic Variants

In various examples, enzymatic converted DNA is used to infer methylation states of C residues in the genome. However, because enzymatic conversion of DNA converts unmethylated C residues to U residues and does not introduce other chemical changes into the DNA, somatic variants that do not correspond to C or T bases in the reference or query sequences can also be identified in the converted DNA. These somatic variants can be identified using existing methods designed for unconverted DNA.

### G. Inferring Nucleosome Positioning

Methylation of cytosine at CpG sites can be greatly enriched in nucleosome-spanning DNA compared to flanking DNA. Therefore, CpG methylation patterns may also be employed to infer nucleosomal positioning using a machine learning approach. The EM-seq datasets may also be analyzed according to the same methods used for WGS to generate features for processing by machine learning methods and models regardless of methylation conversion. Subsequently, 5mC patterns can be used to predict nucleosome positioning, which may aid in inferring gene expression and/or classification of disease and cancer. In another example, features may be obtained from a combination of methylation state and nucleosome positioning information.

Metrics that are used in methylation analysis include, but are not limited to, M-bias (base wise methylation % for CpG, CHG, CHH), conversion efficiency (e.g., 100-Mean methylation % for CHH), hypomethylated blocks, methylation levels (e.g., global mean methylation for CPG, CHH, CHG, chrM, LINE1, or ALU), dinucleotide coverage (normalized coverage of di-nucleotide), evenness of coverage (e.g., unique CpG sites at 1× and 10× mean genomic coverage (for S4 runs), mean CpG coverage (depth) globally, mean coverage at CpG islands, CGI shelves, and CGI shores. In one example, fragment endpoint and length information are used as feature inputs for analysis. These metrics may be used as feature inputs for machine learning methods and models.

In another aspect, the present disclosure provides a method, comprising: (a) providing a biological sample comprising cfDNA from a subject; (b) subjecting the cfDNA to conditions sufficient for optional enrichment of methylated cfDNA in the sample; (c) enzymatically converting unmethylated cytosine nucleobases of the cfDNA into uracil nucleobases; (d) sequencing the cfDNA, thereby generating sequence reads; (e) computer processing the sequence reads to (i) determine a degree of methylation of the cfDNA based on a presence of the uracil nucleobases; and (ii) model the at least partial degradation of the cfDNA, thereby generating degradation parameters; and (f) using the degradation parameters and the degree of methylation to determine a genetic sequence feature.

In some examples, sequencing of cfDNA comprises determining a degree of methylation of the DNA based on a ratio of unconverted cytosine nucleobases to converted cytosine nucleobases. In some examples, the converted cytosine nucleobases are detected as uracil nucleobases. In some examples, the uracil nucleobases are observed as thymine nucleobases in sequence reads.

In some examples, generating degradation parameters comprises using a Bayesian model. In some examples, the Bayesian model is based on strand bias or enzymatic conversion or over-conversion. In some examples, computer processing of the sequence reads comprises using the degradation parameters under the framework of a paired HMM or Naïve Bayesian model.

### H. Analyzing Differentially Methylated Regions (DMRs)

In one example, the methylation analysis is differentially methylated region (DMR) analysis. DMRs are used to quantitate CpG methylation over regions of the genome. The regions are dynamically assigned by discovery. A number of samples from different classes can be analyzed and regions that are the most differentially methylated between the different classifications can be identified. A subset of regions may be selected to be differentially methylated and used for classification. The number of CpGs captured in the region may be used for the analysis. The regions may be variable in size. In one example, a pre-discovery process is performed that bundles a number of CpG sites together as a region. In one example, DMRs are used as input features for processing by machine learning methods and models.

### I. Methylation Haplotype Blocks and Methylation Haplotype Load

In one example, a haplotype block assay is applied to the samples. Identification of methylation haplotype blocks aids in deconvolution of heterogeneous tissue samples and tumor tissue-of-origin mapping from plasma DNA. Tightly coupled CpG sites, referred to as methylation haplotype blocks (MHBs), can be identified in WGBS data. A metric called methylation haplotype load (MHL) is used to perform tissue-specific methylation analysis at the block level. This method provides informative blocks useful for deconvolution of heterogeneous samples. This method is useful for quantitative estimation of tumor load and tissue-of-origin mapping in circulating cfDNA. In one example, haplotype blocks are used as input features for processing by machine learning methods and models.

### J. Targeted Methylation Calling Analysis for Identifying Cell-Type of Origin

In one aspect, methods are used for targeted methylation calling to identify cell-type of origin for cfDNA molecules based on methylation patterns. The method provides a probabilistic model of the joint methylation states of multiple adjacent CpG sites on an individual sequencing read to exploit the pervasive nature of DNA methylation for signal amplification. The model develops a probability of sequencing reads for each cell type and then develops a mixture model for global cell types and fitting to the model.

Traditional DNA methylation analysis focuses on the methylation rate (β-value) of an individual CpG site in a cell population to indicate the proportion of cells in which the CpG site is methylated. Such population-average measures are often not sensitive enough to capture an abnormal methylation signal that affects a small proportion of the cfDNAs. However, based on the pervasive nature of DNA methylation, disease specific cfDNA reads can be computationally differentiated from normal cfDNA reads.

Additionally, given the pervasive nature of DNA methylation, the joint methylation states of multiple adjacent CpG sites may be used to easily distinguish cancer-specific cfDNA reads from normal cfDNA reads. The average of methylation values of all CpG sites in a given read (denoted α-value), provides a difference (0 and 1) between the abnormally methylated cfDNAs and the normal cfDNAs (αtumor=0% and αnormal=100%). The methylation α-value is used to estimate whether the joint probability of all CpG sites in a read follows the DNA methylation signature of a disease. This method can sensitively identify multiple cell-types of origin cfDNAs out of all cfDNAs in plasma.

In various examples, alignment tools are used to align the reads to a reference genome and call the methylated cytosines. PCR duplicates are removed and the numbers of methylated and unmethylated cytosines are quantitated for each CpG site. The methylation level of a CpG cluster is calculated as the ratio between the number of methylated cytosines and the total number of cytosines within the cluster. This WGBS data processing procedure calculates the average methylation level of a CpG cluster in normal plasma samples that are used for identifying methylation markers. When a plasma cfDNA sample is used as test data, the joint methylation-status of all CpG sites of individual sequencing reads that are aligned to the regions of the marker panel is extracted and then processed by a machine learning model. In this approach, the CpG methylation calls are used as input features for methylation state analysis and feature generation. To improve the input data quality for the cfDNA methylation data with high coverage, reads covering <2, <3, or <4 CpG sites can be filtered out.

The methylation sequencing methods described herein improve the sequencing read quality, for example, by reducing PCR errors and bias, and reducing degradation of DNA that occurs with bisulfite conversion. In one example, the methylation sequencing data is used to model overlapping regions. In one example, machine learning modeling can determine cell type-of-origin for identified methylated DNA regions.

In various examples, the model can categorize more than two cell types-of-origin. In other examples, the model can categorize sequences to 3, 4, 5, 6, 7, 8, 9, 10, 15, 20, 50, 75, 100, or more than 100 different cell types.

### K. DNA Hydroxymethylation Analysis

In one aspect of the inventive concepts, 5hmC sequencing can be accomplished by substituting hydroxymethylation in the adapter nucleic acid at the adapter ligation operation, and then only using βGT to conjugate glucose to 5hmC residues in the test nucleic acid library inserts instead of using dioxygenase and βGT to conjugate 5mC and 5hmC. When the resulting sequencing data is compared to a reference genome, every C location in the reference that shows a corresponding C in the test sequence is interpreted as a hydroxymethylated C, and every C in the reference that shows as a T in the test sequence is interpreted as an unmodified C or methylated C. Thus, the data interpretation for hydroxymethylation analysis is the same as for methylation analysis.

In one aspect of the inventive concepts, methylation and hydroxymethylation sequencing libraries can be compared to specify the level of each cytosine modification (e.g., 5m or 5mC) at single nucleotide resolution.

In one aspect of the inventive concepts, since the hydroxymethylation status readout is the same as for methylation status, all analytical methods used with methylation sequencing data can be applied to hydroxymethylation sequencing data.

### III. COMPUTER SYSTEMS AND MACHINE LEARNING METHODS

### A. Sample Features

As used herein, as it relates to machine learning and pattern recognition the term “feature” may refer to an individual measurable property or characteristic of a phenomenon being observed. Features are usually numeric, but structural features such as strings and graphs are used in syntactic pattern recognition. The concept of “feature” is related to that of explanatory variable used in statistical techniques such as linear regression.

In one embodiment, the features are processed into a feature matrix for machine learning analysis.

For a plurality of assays, the system identifies feature sets for processing using a machine learning model. The system performs an assay on each molecule class and forms a feature vector from the measured values. The system processes the feature vector using the machine learning model and obtains an output classification of whether the biological sample has a specified property.

In one embodiment, the machine learning model outputs a classifier that distinguishes between two groups or classes of individuals or features in a population of individuals or features of the population. In one embodiment, the classifier is a trained machine learning classifier.

In one embodiment, the informative loci or features of biomarkers in a cancer tissue are assayed to form a profile. Receiver Operating Characteristic (ROC) curves are useful for plotting the performance of a particular feature (e.g., any of the biomarkers described herein and/or any item of additional biomedical information) in distinguishing between two populations (e.g., individuals responding and not responding to a therapeutic agent). In some embodiments, the feature data across the entire population (e.g., the cases and controls) are sorted in ascending order based on the value of a single feature.

In some embodiments, the condition is advanced adenoma (AA), colorectal cancer (CRC), colorectal carcinoma, or inflammatory bowel disease.

The term “input features” or “features” refers to variables that are used by the model to predict an output classification (label) of a sample, e.g., a condition, sequence content (e.g., mutations), suggested data collection operations, or suggested treatments. Values of the variables can be determined for a sample and used to determine a classification. Example of input features of genetic data include: aligned variables that relate to alignment of sequence data (e.g., sequence reads) to a genome and non-aligned variables, e.g., that relate to the sequence content of a sequence read, a measurement of protein or autoantibody, or the mean methylation level at a genomic region.

Values of the variables can be determined for a sample and used to determine a classification. Example of input features of genetic data include: aligned variables that relate to alignment of sequence data (e.g., sequence reads) to a genome and non-aligned variables, e.g., that relate to the sequence content of a sequence read, a measurement of protein or autoantibody, or the mean methylation level at a genomic region. In various examples, genetic features such as, V-plot measures, FREE-C, the cfDNA measurement over a transcription start site and DNA methylation levels over cfDNA fragments are used as input features for machine learning methods and models.

In one example, the sequencing information includes information regarding a plurality of genetic features such as, but not limited to, transcription start sites, transcription factor binding sites, chromatin open and closed states, nucleosomal positioning or occupancy, and the like.

### B. Data Analysis

In some embodiments, the present disclosure provides a system, method, or kit having data analysis realized in software applications, computing hardware, or both. In various embodiments, the analysis application or system includes at least a data receiving module, a data pre-processing module, a data analysis module (which can operate on one or more types of genomic data), a data interpretation module, or a data visualization module. In one embodiment, the data receiving module can comprise computer systems that connect laboratory hardware or instrumentation with computer systems that process laboratory data. In one embodiment, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

In various embodiments, machine learning methods are applied to distinguish samples in a population of samples. In one embodiment, machine learning methods are applied to distinguish samples between healthy and advanced adenoma samples.

In one embodiment, the one or more machine learning operations used to train the methylation-based prediction engine include one or more of: a generalized linear model, a generalized additive model, a non-parametric regression operation, a random forest classifier, a spatial regression operation, a Bayesian regression model, a time series analysis, a Bayesian network, a Gaussian network, a decision tree learning operation, an artificial neural network, a recurrent neural network, a reinforcement learning operation, linear/non-linear regression operations, a support vector machine, a clustering operation, and a genetic algorithm operation.

In various embodiments, computer processing methods are selected from logistic regression, multiple linear regression (MLR), dimension reduction, partial least squares (PLS) regression, principal component regression, autoencoders, variational autoencoders, singular value decomposition, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, and artificial neural networks.

In some embodiments, the methods disclosed herein can include computational analysis on nucleic acid sequencing data of samples from an individual or from a plurality of individuals. An analysis can identify a variant inferred from sequence data to identify sequence variants based on probabilistic modeling, statistical modeling, mechanistic modeling, network modeling, or statistical inferences. Non-limiting examples of analysis methods include principal component analysis, autoencoders, singular value decomposition, Fourier bases, wavelets, discriminant analysis, regression, support vector machines, tree-based methods, networks, matrix factorization, and clustering. Non-limiting examples of variants include a germline variation or a somatic mutation. In some embodiments, a variant can refer to an already-known variant. The already-known variant can be scientifically confirmed or reported in literature. In some embodiments, a variant can refer to a putative variant associated with a biological change. A biological change can be known or unknown. In some embodiments, a putative variant can be reported in literature, but not yet biologically confirmed.

Alternatively, a putative variant is not reported in literature, but can be inferred based on a computational analysis disclosed herein. In some embodiments, germline variants can refer to nucleic acids that induce natural or normal variations.

Natural or normal variations can include, for example, skin color, hair color, and normal weight. In some embodiments, somatic mutations can refer to nucleic acids that induce acquired or abnormal variations. Acquired or abnormal variations can include, for example, cancer, obesity, conditions, symptoms, diseases, and disorders. In some embodiments, the analysis can include distinguishing between germline variants. Germline variants can include, for example, private variants and somatic mutations. In some embodiments, the identified variants can be used by clinicians or other health professionals to improve health care methodologies, accuracy of diagnoses, and cost reduction.

Also provided herein are improved methods and computing systems or software media that can distinguish among sequence errors in nucleic acid introduced through amplification and/or sequencing techniques, somatic mutations, and germline variants. Methods provided can include simultaneously calling and scoring variants from aligned sequencing data of all samples obtained from a patient.

Samples obtained from subjects other than the patient can also be used. Other samples can also be collected from subjects previously analyzed by a sequencing assay or a targeted sequencing assay (e.g., a targeted resequencing assay). Methods, computing systems, or software media disclosed herein can improve identification and accuracy of variations or mutations (e.g., germline or somatic, including copy number variations, single nucleotide variations, indels, a gene fusions), and lower limits of detection by reducing the number of false positive and false negative identifications.

### C. Classifier Generation

In one aspect, the present systems and methods provide a classifier generated based on feature information derived from methylation sequence analysis from biological samples of cfDNA prepared by the ssDNA library preparation methods described herein. The classifier forms part of a predictive engine for distinguishing groups in a population based on methylation sequence features identified in biological samples such as cfDNA.

In one embodiment, a classifier is created by normalizing the methylation information by formatting similar portions of the methylation information into a unified format and a unified scale; storing the normalized methylation information in a columnar database; training a methylation prediction engine by applying one or more one machine learning operations to the stored normalized methylation information, the methylation prediction engine mapping, for a particular population, a combination of one or more features; applying the methylation prediction engine to the accessed field information to identify a methylation associated with a group; and classifying the individual into a group.

Specificity may be defined as the probability of a negative test among those who are free from the disease. Specificity is equal to the number of disease-free persons who tested negative divided by the total number of disease-free individuals.

In various embodiments, the model, classifier, or predictive test has a specificity of at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 97%, at least 98%, or at least 99%.

Sensitivity may be defined as the probability of a positive test among those who have the disease. Sensitivity is equal to the number of diseased individuals who tested positive divided by the total number of diseased individuals.

In various embodiments, the model, classifier, or predictive test has a sensitivity of at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 97%, at least 98%, or at least 99%.

In one embodiment, the group is selected from healthy (asymptomatic), cancer, gut-associated diseases, immune-mediated inflammatory diseases, neurological diseases, kidney diseases, prenatal diseases, and metabolic diseases.

### D. Digital Processing Device

In some embodiments, the subject matter described herein can include a digital processing device or use of the same. In some embodiments, the digital processing device can include one or more hardware central processing units (CPU), graphics processing units (GPU), or tensor processing units (TPU) that carry out the device's functions. In some embodiments, the digital processing device can include an operating system configured to perform executable instructions. In some embodiments, the digital processing device can optionally be connected a computer network. In some embodiments, the digital processing device can be optionally connected to the Internet such that it accesses the World Wide Web. In some embodiments, the digital processing device can be optionally connected to a cloud computing infrastructure. In some embodiments, the digital processing device can be optionally connected to an intranet. In some embodiments, the digital processing device can be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations.

In some embodiments, the digital processing device can include an operating system configured to perform executable instructions. For example, the operating system can include software, including programs and data, which manages the device's hardware and provides services for execution of applications. Non-limiting examples of operating systems include Ubuntu, FreeBSD, OpenBSD, NetBSD®, Linux, Apple® Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some embodiments, the operating system can be provided by cloud computing, and cloud computing resources can be provided by one or more service providers.

In some embodiments, the device can include a storage and/or memory device. The storage and/or memory device can be one or more physical apparatuses used to store data or programs on a temporary or permanent basis. In some embodiments, the device can be volatile memory and require power to maintain stored information. In some embodiments, the device can be non-volatile memory and retain stored information when the digital processing device is not powered. In some embodiments, the non-volatile memory can include flash memory. In some embodiments, the non-volatile memory can include dynamic random-access memory (DRAM). In some embodiments, the non-volatile memory can include ferroelectric random access memory (FRAM). In some embodiments, the non-volatile memory can include phase-change random access memory (PRAM). In some embodiments, the device can be a storage device including, for example, CD-ROMs, DVDs, flash memory devices, magnetic disk drives, magnetic tapes drives, optical disk drives, and cloud computing-based storage. In some embodiments, the storage and/or memory device can be a combination of devices such as those disclosed herein.

In some embodiments, the digital processing device can include a display to send visual information to a user. In some embodiments, the display can be a cathode ray tube (CRT). In some embodiments, the display can be a liquid crystal display (LCD). In some embodiments, the display can be a thin film transistor liquid crystal display (TFT-LCD). In some embodiments, the display can be an organic light emitting diode (OLED) display. In some embodiments, on OLED display can be a passive-matrix OLED (PMOLED) or active-matrix OLED (AMOLED) display. In some embodiments, the display can be a plasma display. In some embodiments, the display can be a video projector. In some embodiments, the display can be a combination of devices such as those disclosed herein.

In some embodiments, the digital processing device can include an input device to receive information from a user. In some embodiments, the input device can be a keyboard. In some embodiments, the input device can be a pointing device including, for example, a mouse, trackball, track pad, joystick, game controller, or stylus. In some embodiments, the input device can be a touch screen or a multi-touch screen. In some embodiments, the input device can be a microphone to capture voice or other sound input. In some embodiments, the input device can be a video camera to capture motion or visual input. In some embodiments, the input device can be a combination of devices such as those disclosed herein.

### E. Non-Transitory Computer-Readable Storage Medium

In some embodiments, the subject matter disclosed herein can include one or more non-transitory computer-readable storage media encoded with a program including instructions executable by the operating system of an optionally networked digital processing device. In some embodiments, a computer-readable storage medium can be a tangible component of a digital processing device. In some embodiments, a computer-readable storage medium can be optionally removable from a digital processing device. In some embodiments, a computer-readable storage medium can include, for example, CD-ROMs, DVDs, flash memory devices, solid state memory, magnetic disk drives, magnetic tape drives, optical disk drives, cloud computing systems and services, and the like. In some embodiments, the program and instructions can be permanently, substantially permanently, semi-permanently, or non-transitorily encoded on the media.

### F. Computer Systems

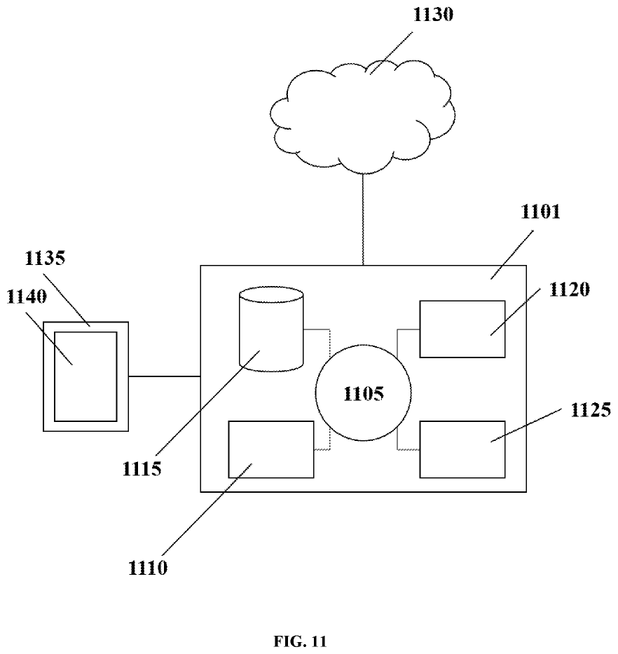

The present disclosure provides computer systems that are programmed to implement methods of the disclosure. FIG. 11 shows a computer system 1101 that is programmed or otherwise configured to store, process, identify, or interpret patient data, biological data, biological sequences, or reference sequences. The computer system 1101 can process various aspects of patient data, biological data, biological sequences, or reference sequences of the present disclosure. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120, and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some embodiments is a telecommunication and/or data network. The network 1130 can include one or more computer servers, which can enable distributed computing, such as cloud computing. The network 1130, in some embodiments with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some embodiments, the circuit is an application specific integrated circuit (ASIC).

The storage unit 1115 can store files, such as drivers, libraries, and saved programs. The storage unit 1115 can store user data, e.g., user preferences and user programs. The computer system 1101 in some embodiments can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some embodiments, the code can be retrieved from the storage unit 1115 and stored on the memory 1110 for ready access by the processor 1105. In some embodiments, the electronic storage unit 1115 can be precluded, and machine-executable instructions are stored on memory 1110.

The code can be pre-compiled and configured for use with a machine having a processer adapted to execute the code or can be interpreted or compiled during runtime. The code can be supplied in a programming language that can be selected to enable the code to execute in a pre-compiled, interpreted, or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as the main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD, or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, a nucleic acid sequence, an enriched nucleic acid sample, an expression profile, and an analysis of an expression profile. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, probe a plurality of regulatory elements, sequence a nucleic acid sample, enrich a nucleic acid sample, determine an expression profile of a nucleic acid sample, analyze an expression profile of a nucleic acid sample, and archive or disseminate results of analysis of an expression profile.

In some embodiments, the subject matter disclosed herein can include at least one computer program or use of the same. A computer program can be a sequence of instructions, executable in the digital processing device's CPU, GPU, or TPU, written to perform a specified task. Computer-readable instructions can be implemented as program modules, such as functions, objects, Application Programming Interfaces (APIs), data structures, and the like, that perform particular tasks or implement particular abstract data types. A computer program can be written in various versions of various languages.

The functionality of the computer-readable instructions can be combined or distributed as desired in various environments. In some embodiments, a computer program can include one sequence of instructions. In some embodiments, a computer program can include a plurality of sequences of instructions. In some embodiments, a computer program can be provided from one location. In some embodiments, a computer program can be provided from a plurality of locations. In some embodiments, a computer program can include one or more software modules. In some embodiments, a computer program can include, in part or in whole, one or more web applications, one or more mobile applications, one or more standalone applications, one or more web browser plug-ins, extensions, add-ins or add-ons, or combinations thereof.

In some embodiments, computer processing can be a method of statistics, mathematics, biology, or any combination thereof. In some embodiments, the computer processing method includes a dimension reduction method including, for example, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, and neural network.

In some embodiments, the computer processing method is a supervised machine learning method including, for example, a regression, support vector machine, tree-based method, and network.

In some embodiments, the computer processing method is an unsupervised machine learning method including, for example, clustering, network, principal component analysis, and matrix factorization.

### G. Databases

In some embodiments, the subject matter disclosed herein can include one or more databases, or use of the same to store patient data, biological data, biological sequences, or reference sequences. Reference sequences can be derived from a database. Various databases can be suitable for storage and retrieval of the sequence information. In some embodiments, suitable databases can include, for example, relational databases, non-relational databases, object-oriented databases, object databases, entity-relationship model databases, associative databases, and XML databases. In some embodiments, a database can be internet-based. In some embodiments, a database can be web-based. In some embodiments, a database can be cloud computing-based. In some embodiments, a database can be based on one or more local computer storage devices.

### IV. CANCER DETECTION AND DIAGNOSIS

The trained machine learning methods, models, and discriminate classifiers described herein are useful for various medical applications including cancer detection, diagnosis, and treatment responsiveness. As models are trained with individual metadata and analyte-derived features, the applications may be tailored to stratify individuals in a population and guide treatment decisions accordingly.

### A. Detection/Diagnosis

Methods and systems provided herein may perform predictive analytics using artificial intelligence-based approaches to analyze acquired data from a subject (patient) to generate an output of the detection and/or diagnosis of a subject having a cancer (e.g., CRC) or other indications. For example, the application may apply a prediction algorithm to the acquired data to generate the detection of cancer thereby providing a diagnosis that the subject has cancer. The prediction algorithm may comprise an artificial intelligence-based predictor, such as a machine learning-based predictor, configured to process the acquired data to generate the diagnosis of the subject having the cancer.

The machine learning predictor may be trained using datasets, e.g., datasets generated by performing multi-analyte assays of biological samples of individuals, from one or more sets of cohorts of patients having cancer as inputs and known diagnosis (e.g., staging and/or tumor fraction) outcomes of the subjects as outputs to the machine learning predictor.

Training datasets (e.g., datasets generated by performing multi-analyte assays of biological samples of individuals) may be generated from, for example, one or more sets of subjects having common characteristics (features) and outcomes (labels). Training datasets may comprise a set of features and labels corresponding to the features relating to diagnosis. Features may comprise characteristics such as, for example, certain ranges or categories of cfDNA assay measurements, such as counts of cfDNA fragments in a biological sample obtained from a healthy and disease samples that overlap or fall within each of a set of bins (genomic windows) of a reference genome. For example, a set of features collected from a given subject at a given time point may collectively serve as a diagnostic signature, which may be indicative of an identified cancer of the subject at the given time point. Characteristics may also include labels indicating the subject's diagnostic outcome, such as for one or more cancers.

Labels may comprise outcomes such as, for example, a known diagnosis (e.g., staging and/or tumor fraction) outcomes of the subject. Outcomes may include a characteristic associated with the cancers in the subject. For example, characteristics may be indicative of the subject having one or more cancers.

Training sets (e.g., training datasets) may be selected by random sampling of a set of data corresponding to one or more sets of subjects (e.g., retrospective and/or prospective cohorts of patients having or not having one or more cancers). Alternatively, training sets (e.g., training datasets) may be selected by proportionate sampling of a set of data corresponding to one or more sets of subjects (e.g., retrospective and/or prospective cohorts of patients having or not having one or more cancers). Training sets may be balanced across sets of data corresponding to one or more sets of subjects (e.g., patients from different clinical sites or trials). The machine learning predictor may be trained until certain predetermined conditions for accuracy or performance are satisfied, such as having minimum desired values corresponding to diagnostic accuracy measures. For example, the diagnostic accuracy measure may correspond to prediction of a diagnosis, staging, or tumor fraction of one or more cancers in the subject.

Examples of detection and diagnostic accuracy measures may include sensitivity, specificity, positive predictive value (PPV), negative predictive value (NPV), accuracy, and area under the curve (AUC) of a ROC curve corresponding to the diagnostic accuracy of detecting or predicting the cancer (e.g., colorectal cancer).

In another aspect, the present disclosure provides a method for detecting or identifying a cancer in a subject, comprising: (a) providing a biological sample comprising ssDNA molecules of a cell-free DNA sample derived from said subject; (b) methylation sequencing said ssDNA molecules from said subject to generate a plurality of sequencing reads; (c) aligning said sequencing reads to a reference genome; (d) generating a quantitative measure of said sequencing reads at each of a first plurality of genomic regions of said reference genome to generate a first feature set, wherein said first plurality of genomic regions of said reference genome comprises at least about 10 distinct regions, each of said at least about 10 distinct regions; and (e) applying a trained algorithm to said first feature set to generate a likelihood of said subject having said cancer.

For example, such a predetermined condition may be that the sensitivity of predicting the cancer (e.g., colorectal cancer, breast cancer, pancreatic cancer, liver, or lung cancer) comprises a value of, for example, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

As another example, such a predetermined condition may be that the specificity of predicting the cancer (e.g., colorectal cancer, breast cancer, pancreatic cancer, liver, or lung cancer) comprises a value of, for example, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

As another example, such a predetermined condition may be that the positive predictive value (PPV) of predicting the cancer (e.g., colorectal cancer, breast cancer, pancreatic cancer, liver, or lung cancer) comprises a value of, for example, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

As another example, such a predetermined condition may be that the negative predictive value (NPV) of predicting the cancer (e.g., colorectal cancer, breast cancer, pancreatic cancer, liver or lung cancer) comprises a value of, for example, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

As another example, such a predetermined condition may be that the AUC of a ROC curve of predicting the cancer (e.g., colorectal cancer, breast cancer, pancreatic cancer, liver or lung cancer) comprises a value of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, or at least about 0.99.

In some examples of any of the foregoing aspects, a method further comprises monitoring a progression of a disease in the subject, wherein the monitoring is based at least in part on the genetic sequence feature. In some examples, the disease is a cancer.

In some examples of any of the foregoing aspects, a method further comprises determining the tissue-of-origin of a cancer in the subject, wherein the determining is based at least in part on the genetic sequence feature.

In some examples of any of the foregoing aspects, a method further comprises estimating a tumor burden in the subject, wherein the estimating is based at least in part on the genetic sequence feature.

### B. Treatment Responsiveness

The predictive classifiers, systems and methods described herein are useful for classifying populations of individuals for a number of clinical applications (e.g., based on performing multi-analyte assays of biological samples of individuals). Examples of such clinical applications include, detecting early-stage cancer, diagnosing cancer, classifying cancer to a particular stage of disease, or determining responsiveness or resistance to a therapeutic agent for treating cancer.

The methods and systems described herein are applicable to various cancer types, similar to grade and stage, and as such, is not limited to a single cancer disease type. Therefore, combinations of analytes and assays may be used in the present systems and methods to predict responsiveness of cancer therapeutics across different cancer types in different tissues and classifying individuals based on treatment responsiveness. In one example, the classifiers described herein stratify a group of individuals into treatment responders and non-responders.

The present disclosure also provides a method for determining a drug target of a condition or disease of interest (e.g., genes that are relevant/important for a particular class), comprising assessing a sample obtained from an individual for the level of gene expression for at least one gene; using a neighborhood analysis method to determine genes that are relevant for classification of the sample, thereby ascertaining one or more drug targets relevant to the classification.

The present disclosure also provides a method for determining the efficacy of a drug designed to treat a disease class, comprising obtaining a sample from an individual having the disease class; subjecting the sample to the drug; assessing the drug-exposed sample for the level of gene expression for at least one gene; and using a computer model built with a weighted voting scheme to classify the drug-exposed sample into a class of the disease as a function of relative gene expression level of the sample with respect to that of the model.

The present disclosure also provides a method for determining the efficacy of a drug designed to treat a disease class, wherein an individual has been subjected to the drug, comprising obtaining a sample from the individual subjected to the drug; assessing the sample for the level of gene expression for at least one gene; and using a model built with a weighted voting scheme to classify the sample into a class of the disease including evaluating the gene expression level of the sample as compared to gene expression level of the model.

Yet another application is a method of determining whether an individual belongs to a phenotypic class (e.g., intelligence, response to a treatment, length of life, likelihood of viral infection or obesity) that comprises obtaining a sample from the individual; assessing the sample for the level of gene expression for at least one gene; and using a model built with a weighted voting scheme, classifying the sample into a class of the disease including evaluating the gene expression level of the sample as compared to gene expression level of the model.

Biomarkers may be useful for predicting prognosis of patients with colon cancer. The ability to classify patients as high-risk (poor prognosis) or low-risk (favorable prognosis) may enable selection of appropriate therapies for these patients. For example, high-risk patients are likely to benefit from aggressive therapy, whereas therapy may have no significant advantage for low-risk patients.

Predictive biomarkers that can guide treatment decisions by identifying subsets of patients who may be “exceptional responders” to specific cancer therapies, or individuals who may benefit from alternative treatment modalities.

In one aspect, the systems and methods described herein that relate to classifying a population based on treatment responsiveness refer to cancers that are treated with chemotherapeutic agents of the classes DNA damaging agents, DNA repair target therapies, inhibitors of DNA damage signaling, inhibitors of DNA damage induced cell cycle arrest, and inhibition of processes indirectly leading to DNA damage, but not limited to these classes. Each of these chemotherapeutic agents may be considered a “DNA-damage therapeutic agent”.

The patient's analyte data are classified in high-risk and low-risk patient groups, such as patient with a high-risk or low-risk of clinical relapse, and the results may be used to determine a course of treatment. For example, a patient determined to be a high-risk patient may be treated with adjuvant chemotherapy after surgery. For a patient deemed to be a low-risk patient, adjuvant chemotherapy may be withheld after surgery. Accordingly, the present disclosure provides, in certain aspects, a method for preparing a gene expression profile of a colon cancer tumor that is indicative of risk of recurrence.

In various examples, the classifiers described herein stratify a population of individuals between responders and non-responders to treatment.

In various examples, the treatment is selected from alkylating agents, plant alkaloids, antitumor antibiotics, antimetabolites, topoisomerase inhibitors, retinoids, checkpoint inhibitor therapy, and VEGF inhibitors.

Examples of treatments for which a population may be stratified into responders and non-responders include but are not limited to: chemotherapeutic agents including sorafenib, regorafenib, imatinib, eribulin, gemcitabine, capecitabine, pazopanib, lapatinib, dabrafenib, sunitinib, crizotinib, everolimus, torisirolimus, sirolimus, axitinib, gefitinib, anastrozole, bicalutamide, fulvestrant, raltitrexed, pemetrexed, goserelin acetate, erlotinib, vemurafenib, vismodegib, tamoxifen citrate, paclitaxel, docetaxel, cabazitaxel, oxaliplatin, ziv-aflibercept, bevacizumab, trastuzumab, pertuzumab, panitumumab, taxane, bleomycin, melphalan, plumbagin, camptosar, mitomycin-C, mitoxantrone, poly(styrene-maleic acid)-conjugated neocarzinostatin (SMANCS), doxorubicin, pegylated doxorubicin, FOLFORI, 5-fluorouracil, temozolomide, pasireotide, tegafur, gimeracil, oteracil, itraconazole, bortezomib, lenalidomide, irinotecan, epirubicin, romidepsin, resminostat, tasquinimod, refametinib, lapatinib, Tyverb®, Arenegyr, NGR-TNF, pasireotide, Signifor®, ticilimumab, tremelimumab, lansoprazole, PrevOnco®, ABT-869, linifanib, vorolanib, tivantinib, Tarceva®, erlotinib, Stivarga®, regorafenib, fluoro-sorafenib, brivanib, liposomal doxorubicin, lenvatinib, ramucirumab, peretinoin, muparfostat, Teysuno®, tegafur, gimeracil, oteracil, and orantinib; and antibody therapies, including but not limited to, alemtuzumab, atezolizumab, ipilimumab, nivolumab, ofatumumab, pembrolizumab, or rituximab.

In other examples, a population may be stratified into responders and non-responders for checkpoint inhibitor therapies such as compounds that bind to PD-1 or CTLA4.

In other examples, a population may be stratified into responders and non-responders for anti-VEGF therapies that bind to VEGF pathway targets.

### V. INDICATIONS

In some examples, a biological condition can include a disease. In some examples, a biological condition can be a stage of a disease. In some examples, a biological condition can be a gradual change of a biological state. In some examples, a biological condition can be a treatment effect. In some examples, a biological condition can be a drug effect. In some examples, a biological condition can be a surgical effect. In some examples, a biological condition can be a biological state after a lifestyle modification. Non-limiting examples of lifestyle modifications include a diet change, a smoking change, and a sleeping pattern change. In some examples, a biological condition is unknown. The analysis described herein can include machine learning to infer an unknown biological condition or to interpret the unknown biological condition.

In one example, the present systems and methods are particularly useful for applications related to colon cancer: Cancer that forms in the tissues of the colon (the longest part of the large intestine). Most colon cancers are adenocarcinomas (cancers that begin in cells that make line internal organs and have gland-like properties). Cancer progression is characterized by stages, or the extent of cancer in the body. Staging is usually based on the size of the tumor, whether lymph nodes contain cancer, and whether the cancer has spread from the original site to other parts of the body. Stages of colon cancer include stage I, stage II, stage III, and stage IV. Unless otherwise specified, the term “colon cancer” refers to colon cancer at Stage 0, Stage I, Stage II (including Stage IIA or IIB), Stage III (including Stage IIIA, IIIB, or IIIC), or Stage IV. In some examples herein, the colon cancer is from any stage. In one example, the colon cancer is a stage I colorectal cancer. In one example, the colon cancer is a stage II colorectal cancer. In one example, the colon cancer is a stage III colorectal cancer. In one example, the colon cancer is a stage IV colorectal cancer.

Conditions that can be inferred by the disclosed methods include, for example, cancer, gut-associated diseases, immune-mediated inflammatory diseases, neurological diseases, kidney diseases, prenatal diseases, and metabolic diseases.

In some examples, a method of the present disclosure can be used to diagnose a cancer. Non-limiting examples of cancers include adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

Non-limiting examples of cancers that can be inferred by the disclosed methods and systems include acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloepithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom macroglobulinemia, and Wilms Tumor.

Non-limiting examples of gut-associated diseases that can be inferred by the disclosed methods and systems include Crohn's disease, colitis, ulcerative colitis (UC), inflammatory bowel disease (IBD), irritable bowel syndrome (IBS), and celiac disease. In some examples, the disease is inflammatory bowel disease, colitis, ulcerative colitis, Crohn's disease, microscopic colitis, collagenous colitis, lymphocytic colitis, diversion colitis, Behçet's disease, and indeterminate colitis.

Non-limiting examples of immune-mediated inflammatory diseases that can be inferred by the disclosed methods and systems include psoriasis, sarcoidosis, rheumatoid arthritis, asthma, rhinitis (hay fever), food allergy, eczema, lupus, multiple sclerosis, fibromyalgia, type 1 diabetes, and Lyme disease. Non-limiting examples of neurological diseases that can be inferred by the disclosed methods and systems include Parkinson's disease, Huntington's disease, multiple sclerosis, Alzheimer's disease, stroke, epilepsy, neurodegeneration, and neuropathy.

Non-limiting examples of kidney diseases that can be inferred by the disclosed methods and systems include interstitial nephritis, acute kidney failure, and nephropathy. Non-limiting examples of prenatal diseases that can be inferred by the disclosed methods and systems include Down syndrome, aneuploidy, spina bifida, trisomy, Edwards syndrome, teratomas, sacrococcygeal teratoma (SCT), ventriculomegaly, renal agenesis, cystic fibrosis, and hydrops fetalis. Non-limiting examples of metabolic diseases that can be inferred by the disclosed methods and systems include cystinosis, Fabry disease, Gaucher disease, Lesch-Nyhan syndrome, Niemann-Pick disease, phenylketonuria, Pompe disease, Tay-Sachs disease.

The specific details of particular examples may be combined in any suitable manner without departing from the spirit and scope of disclosed examples of the inventive concepts. However, other examples of the inventive concepts may be directed to specific examples relating to each individual aspect, or specific combinations of these individual aspects. All patents, patent applications, publications, and descriptions mentioned herein are incorporated by reference in their entirety for all purposes.

### VI. KITS

The present disclosure provides kits for identifying or monitoring a cancer of a subject. A kit may comprise probes for identifying a quantitative measure (e.g., indicative of a presence, absence, or relative amount) of sequences at each of a plurality of cancer-associated genomic loci in a cell-free biological sample of the subject. A quantitative measure (e.g., indicative of a presence, absence, or relative amount) of sequences at each of a plurality of cancer-associated genomic loci in the cell-free biological sample may be indicative of one or more cancers. The probes may be selective for the sequences at the plurality of cancer-associated genomic loci in the cell-free biological sample. A kit may comprise instructions for using the probes to process the cell-free biological sample to generate datasets indicative of a quantitative measure (e.g., indicative of a presence, absence, or relative amount) of sequences at each of the plurality of cancer-associated genomic loci in a cell-free biological sample of the subject. In one embodiment, the kit comprises primer sets, PCR reaction components, sequencing reagents, minimally-destructive conversion reagents, and library preparation reagents.

The probes in the kit may be selective for the sequences at the plurality of cancer-associated genomic loci in the cell-free biological sample. The probes in the kit may be configured to selectively enrich nucleic acid (e.g., RNA or DNA) molecules corresponding to the plurality of cancer-associated genomic loci. The probes in the kit may be nucleic acid primers. The probes in the kit may have sequence complementarity with nucleic acid sequences from one or more of the plurality of cancer-associated genomic loci or genomic regions. The plurality of cancer-associated genomic loci or genomic regions may comprise more than or equal to 2, 3, 4, 5, 6, 7, 8, 9, 10, 11, 12, 13, 14, 15, 16, 17, 18, 19, 20, 21, 22, 23, 24, 25, 26, 27, 28, 29, or 30 distinct cancer-associated genomic loci or genomic regions identified for targeted methylation sequencing. The plurality of cancer-associated genomic loci or genomic regions may comprise less than or equal to 30, 29, 28, 27, 26, 25, 24, 23, 22, 21, 20, 19, 18, 17, 16, 15, 14, 13, 12, 11, 10, 9, 8, 7, 6, 5, 4, 3, or 2 distinct cancer-associated genomic loci or genomic regions identified for targeted methylation sequencing

The instructions in the kit may comprise instructions to assay the cell-free biological sample using the probes that are selective for the sequences at the plurality of cancer-associated genomic loci in the cell-free biological sample. These probes may be nucleic acid molecules (e.g., RNA or DNA) having sequence complementarity with nucleic acid sequences (e.g., RNA or DNA) from one or more of the plurality of cancer-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the cell-free biological sample to generate datasets indicative of a quantitative measure (e.g., indicative of a presence, absence, or relative amount) of sequences at each of the plurality of cancer-associated genomic loci in the cell-free biological sample. A quantitative measure (e.g., indicative of a presence, absence, or relative amount) of sequences at each of a plurality of cancer-associated genomic loci in the cell-free biological sample may be indicative of one or more cancers.

The instructions in the kit may comprise instructions to measure and interpret assay readouts, which may be quantified at one or more of the plurality of cancer-associated genomic loci to generate the datasets indicative of a quantitative measure (e.g., indicative of a presence, absence, or relative amount) of sequences at each of the plurality of cancer-associated genomic loci in the cell-free biological sample. For example, quantification of array hybridization or polymerase chain reaction (PCR) corresponding to the plurality of cancer-associated genomic loci may generate the datasets indicative of a quantitative measure (e.g., indicative of a presence, absence, or relative amount) of sequences at each of the plurality of cancer-associated genomic loci in the cell-free biological sample. Assay readouts may comprise quantitative PCR (qPCR) values, digital PCR (dPCR) values, digital droplet PCR (ddPCR) values, fluorescence values, etc., or normalized values thereof.

### EXAMPLES

### Example 1

### DNA Extraction and Library Preparation for Targeted EM-Seq with Conversion-Tolerant Sequencing Adapter/Primer Systems

### A. DNA Preparation

### Starting Material: 2 mL of Plasma

DNA was extracted from 2 mL of plasma using the QIAamp Circulating Nucleic Acids DNA purification according to the manufacturer's protocol and eluted to a 30 μL volume.

B. Denaturation of the dsDNA to ssDNA

The extracted DNA sample (containing dsDNA) was denatured by incubating the plate at 98° C. for about 3 minutes on a thermocycler to generate ssDNA. Upon removal from the thermocycler, the denatured sample was immediately placed on a frozen plate block on ice.

### C. Conversion-Tolerant Adapter Ligation

In this example, a functional set of conversion tolerant adapters, PCR primers, and sequencing primers was tested as described in Example 2. Sequencing library yield for libraries generated with either conversion-tolerant adapters or 5mC-containing adapters was determined.

Conversion-tolerant single-stranded DNA library adapters including both 5′ and 3′ adapter pairs, referred to as adapter A and adapter B, respectively, were prepared (FIG. 2). Adapter A comprised of a top DNA oligonucleotide with a 5′ terminal amino modifier including a 12-carbon spacer. All cytosines were 5mC in the top adapter. The 3′ sequences of each top adapter contained one of several mCp-containing motifs used for assessment of enzymatic oxidation and deamination performance. Each top A adapter was annealed to a bottom adapter (adapter A′) containing unmethylated cytosines. The bottom adapter strand had a fully complementary sequence to the top adapter strand except for a random 7-nucleotide 5′ overhang sequence. The lower adapter ends were modified with a 5′ amino modifier with a 6-carbon spacer and a 3′ amino modifier. Adapter B pairs comprised of a top DNA oligonucleotide with a 5′ phosphate, followed by one of several mCp-containing motifs, a sequence in which all cytosines are methylated, and a 3′ dideoxycytosine. Each top B adapter was annealed to a bottom adapter (adapter B′) containing unmethylated cytosines. The bottom adapter strand had a fully complementary sequence to the top adapter strand except for a random 7-nucleotide 3′ overhang sequence. All lower B adapter oligonucleotides were blocked with a 5′ terminal amino modifier including a 12-carbon spacer, and a 3′ amino modifier. Alternatively, conversion-tolerant adapters can be generated by excluding all cytosines from the top adapter sequences.

About 20 μL of cfDNA mass input was parsed into a 96-well plate. The plate was sealed, vortexed, and spun down by centrifugation. The samples were then placed into a thermocycler at 98° C. (lid temperature 105° C.) for 3 minutes. Samples were removed from the thermocycler, immediately placed on a frozen plate block on ice, and held for 5 minutes. Subsequently, 2 μL of each of the 5′ and 3′ ssDNA adapter pairs were added to the ligation mix, which was subsequently added to each sample and pipette-mixed ten times. Then, 26 μL of the ligation buffer were added to the samples and pipette-mixed ten times. The plate was sealed, spun down by centrifugation, vortexed, and spun down by centrifugation again. Then, the plate was incubated on a thermocycler at 37° C. (lid temperature 45° C.) for one hour.

After the hour ligation incubation, the plate was removed from the thermocycler for a bead-based cleanup. First, a diluted bead mix was prepared: 75 μL of 10 mM Tris-HCl (pH 8.5) were added to 58.5 μL of previously-diluted, buffer-exchanged AMPure beads. About 133.5 μL of this bead mix were added to each sample. The sample-bead combination was then incubated at room temperature on a benchtop for about 15 minutes. Next, the plate was placed on a Permagen bar magnet for 5 minutes, or until the supernatant became clear in appearance. The supernatant was then removed without disturbing the magnetized bead pellet. While the plate was still on the magnetized rack, 200 μL of freshly prepared 80% ethanol (EtOH) were then added to each well. After 30 seconds, the EtOH was removed. This wash operation was then repeated (200 μL of ethanol added and then removed after 30 seconds). The plate was then removed from the magnet, sealed, and pulse-spun by centrifugation. The plate was returned to the magnet, and the remaining EtOH was removed with a small pipette. The magnetized beads were allowed to dry at room temperature on the benchtop for two minutes, then removed from the magnet. Off-magnet, 16 μL of 10 mM Tris-HCl was added to the beads and pipette-mixed thoroughly. The beads were incubated at room temperature for 5 minutes, then the plate was returned to the magnet. When the supernatant became clear, 15 μL of the supernatant was transferred to a new plate.

### D. Second Strand Synthesis

In an alternate embodiment, a second strand synthesis (SSS) operation may be added to convert ssDNA libraries to dsDNA libraries after adapter ligation and prior to the enzymatic oxidation reaction. Following a ssDNA library preparation, a bead purification cleanup was performed. Then, KAPA PCR master mix, Tris-HCl, and 2 μL of 50 μM primer complementary to the 3′ adapter were added to the libraries. Samples were vortexed and spun down by centrifugation, then subjected to a single extension reaction on a thermocycler. Following this extension, samples were cleaned with purification beads and eluted in an elution buffer.

### E. No Clean-Up Second Strand Synthesis

Following ssDNA library preparation, no cleanup was performed. Immediately after adapter ligation, the following were added to each sample: 1 μL of 50 μM primer complementary to the 3′ adapter, 0.5 μL dNTPs (from Fast Start kit), 0.2 μL Fast Taq polymerase, and water to increase volume. Samples were vortexed and spun by centrifugation, then subjected to an extension on the thermocycler. Following the extension, samples are cleaned with purification beads and eluted in an elution buffer.

F. No Clean-Up Second Strand Synthesis with Alternate Adapters

Single stranded ligation was performed as described above; however, the bottom adapter B sequence was truncated as to not fully complement the top adapter B sequence. Following ssDNA library preparation, no cleanup was performed. Immediately after adapter ligation, the following were added to each sample: 1 μL of 50 μM primer complementary to the 3′ adapter, 0.5 μL dNTPs (from Fast Start kit), 0.2 μL Bst polymerase, and water to increase volume. Samples were vortexed and spun by centrifugation, then underwent one PCR cycle on the thermocycler. Following the extension, samples were cleaned with purification beads and eluted in an elution buffer.

The ssDNA libraries, whether single-stranded or double-stranded (with or without second strand synthesis), with conversion-tolerant adapters were then used as the starting material for the subsequent methylation conversion and sequencing reaction described herein in Example 2.

### Example 2

### Targeted EM-seq Library Preparation

### A. Oxidation of 5-Methylcytosines and 5-Hydroxymethylcytosines

TET2 Reaction Buffer was prepared according to the manufacturer's protocol. The TET2 Reaction Buffer was then added to one tube of TET2 Reaction Buffer Supplement, followed by thorough mixing. On ice, TET2 Reaction Buffer, Oxidation Supplement, Oxidation Enhancer, and TET2 enzyme were added directly to the ssDNA libraries prepared according to Example 1 (“samples”). Each of the sample mixtures was then mixed thoroughly by vortexing. After centrifuging the mixture briefly, an iron solution was added to the mixtures. The mixtures were then mixed thoroughly by vortexing or by pipetting up and down, and centrifuged briefly. The mixtures were then incubated at 37° C. for 1 hour in a thermocycler. The mixtures were then transferred to ice before treating with 1 μl of Stop Reagent and the appearance of the mixtures was yellow. The mixtures were then mixed thoroughly by vortexing or by pipetting up and down at least 10 times and centrifuged briefly. Finally, the mixtures were incubated at 37° C. for 30 minutes, then at 4° C. in a thermocycler.

### B. Clean-Up of TET2 Converted DNA

NEBNext® Sample Purification Beads were vortexed and then added to each sample, followed by thorough mixing by pipetting up and down. The samples were incubated on the bench top for at least 5 minutes at room temperature. The tubes were then placed against an appropriate magnetic stand to separate the beads from the supernatant. After 5 minutes (or when the solution is clear), the supernatant was carefully removed to avoid disturbing the beads that contain DNA targets, and discarded. While on the magnetic stand, freshly prepared 80% ethanol was added to each of the tubes. The samples were incubated at room temperature for 30 seconds before the supernatant was carefully removed and discarded. The wash was repeated once for a total of two washes. All visible liquid was removed after the second wash using a p10 pipette tip. The beads were then air dried for 2 minutes while the tubes are on the magnetic stand with the lid open. The tubes were then removed from the magnetic stand. The DNA was eluted from the beads with Elution Buffer. Elution Buffer was added to each of the tubes and mixed thoroughly by pipetting up and down 10 times. The samples were then incubated for at least 1 minute at room temperature. If necessary, the sample was quickly centrifuged to collect the liquid from the sides of the tube before placing the tubes back on the magnetic stand. The tubes were then placed back on the magnetic stand. After 3 minutes (or whenever the solution is clear), the eluted DNA from the supernatant was transferred to a new PCR tube.

### C. Denaturation of DNA

The DNA sample (containing dsDNA) was denatured by incubating the plate at 85° C. for 10 minutes on a thermocycler. Upon removal from the thermocycler, the DNA sample was immediately placed on a frozen plate block on ice.

### D. Deamination of Cytosines

APOBEC Reaction Buffer, bovine serum albumin (BSA), and APOBEC were added to the denatured DNA. The mixture was then mixed thoroughly by vortexing or by pipetting up and down at least 10 times before centrifuging briefly. The mixture was then incubated according to the following protocol: 4° C. for 10 minutes, then increase 1° C. every 2 minutes and 15 seconds forty-six times until the sample reaches 50° C. Then, hold at 50° C. for 10 minutes, then hold at 4° C. in a thermocycler.

### E. Thermolabile Proteinase K Treatment

To halt the APOBEC activity, Thermolabile Proteinase K (TLPK) was added to the samples after deamination and incubated for 30 minutes at 37° C. for 15 minutes, then inactivated at 65° C. for 10 minutes. Following the first TLPK treatment, samples were amplified via PCR with indexing primers. Amplification can be run for any number of cycles. Following PCR, a second TLPK treatment was performed. Following the second TLPK treatment, a bead purification cleanup was performed.

### F. Clean-Up of Deaminated DNA

NEBNext® Sample Purification Beads were vortexed and then added to each sample, followed by thorough mixing by pipetting up and down at least 10 times. During the last mix, all liquid was carefully expelled out of the tip. The samples were then incubated on the bench top for at least 5 minutes at room temperature. After 5 minutes (or when the solution is clear), the supernatant was carefully removed and discarded. While on the magnetic stand, freshly prepared 80% ethanol was added to the tubes. The samples were then incubated at room temperature for 30 seconds before the supernatant was carefully removed and discarded. The wash was repeated once for a total of two washes. Next, the beads were air dried for 90 seconds while the tubes are on the magnetic stand with the lid open. The DNA targets were then eluted from the beads with Elution Buffer. Elution Buffer was added to each of the tubes and mixed thoroughly by pipetting up and down 10 times. The samples were incubated for at least 1 minute at room temperature. If necessary, the sample was quickly centrifuged to collect the liquid from the sides of the tube before placing the tubes back on the magnetic stand. The tubes were then placed back on the magnetic stand. After 3 minutes (or whenever the solution is clear), the eluted DNA targets in the supernatant were transferred to a new PCR tube.

### Example 3

### Target Capture and Multiplex Amplification

After quantification, the samples were pooled and concentrated. Then, target enrichment was performed on the enzymatic converted libraries to specifically enrich for pre-identified DNA fragments that contain target CpG sites using 5′-biotinylated capture probes. These probes can be methylated, unmethylated, or a combination of both. Hybrid selection was carried out using the TWIST Fast Hybridization Target Capture Kit. Hybridization can occur at 58° C., 60° C., or other temperatures. Bead washes were performed at heated temperatures, e.g., hybridization temperature+3° C. Following hybridization, the captured DNA fragments were amplified with PCR. Target capture libraries were sequenced on an Illumina Novaseq Sequencer using 2×150 cycle runs.

### Example 4

### Targeted Methylation Classification

Raw data files were used for alignment and methylation calling to permit targeted methylation analysis for pre-identified regions of the genome. Whole genome amplification of the enzymatic converted DNA was carried out.

FASTQ files were mapped to a reference genome, and methylation scores were calculated for disease classification. Featurized data comprising a set of CpG sites associated with healthy, disease, disease state, and treatment responsiveness was processed using machine learning models to identify classifiers that stratify individuals in a population based upon hypermethylation model scores and/or hypomethylation model scores.

### Example 5

### Comparison of ssDNA Library Preparation Versus dsDNA Library Preparation in Methylation Studies

### A. Advance Adenoma (AA) and Colorectal Cancer (CRC)

Blood samples were obtained from 11 healthy human subjects, 23 advanced adenoma (AA) human subjects, and 30 colorectal cancer (CRC) human subjects. DNA was extracted from the blood samples and library preparation was performed according to: (i) the ssDNA library preparation methods set forth in Example 1 and (ii) other dsDNA library preparation methods to generate ssDNA libraries and dsDNA libraries respectively (E7120 NEBNext® enzymatic methyl-seq kit E7120).

Both the ssDNA libraries (generated by the methods disclosed herein) and the dsDNA libraries were subject to targeted methylation conversion sequencing methods as described in Examples 2-3. The raw data files were used for alignment and methylation calling to permit targeted methylation analysis for pre-identified regions of the genome for both hypermethylation model scoring and hypomethylation model scoring.

### B. Results

FIG. 5 provides a comparison of hypermethylation model scores of cfDNA extracted from the healthy/cancer-negative (NEG), advance adenoma (AA), and colorectal cancer (CRC) samples detected using ssDNA library preparation described in Example 1 versus another dsDNA library preparation method. The hypermethylation rates of cfDNA derived from selected genomic regions distinguished AA and CRC patient-derived cfDNA from cancer-negative cfDNA when processed using both the ssDNA library preparation and the other dsDNA library preparation. This experiment demonstrates that the ssDNA library preparation methods described herein can yield equivalent methylation analysis (e.g., hypermethylation scores) as other dsDNA library preparation methods. Thus, this workflow is equivalent to end repair-dependent methods at discriminating cancer patient-derived from healthy patient-derived cfDNA samples, e.g., when performing DNA hypermethylation analysis.

FIG. 7 provides a comparison of hypomethylation scores of cfDNA extracted from the healthy/cancer-negative (NEG), advance adenoma (AA), and colorectal cancer (CRC) samples detected using ssDNA library preparation described in Example 1 versus another dsDNA library preparation method. Hypomethylation rates of cfDNA derived from selected genomic regions distinguished AA and CRC patient-derived cfDNA from cancer-negative cfDNA when processed using ssDNA library preparation described herein but not when processed using dsDNA library preparation. Thus, this example demonstrates that the ssDNA library preparation method described herein optimizes the recovery, methylation analysis, quantification, and target capture. As such, in certain embodiments, this workflow can outperform end repair-dependent methods at discriminating cancer patient-derived from healthy patient-derived cfDNA samples, e.g., using DNA hypomethylation analysis.

### C. Liver Cancer, Lung Cancer, and Pancreatic Cancer

Blood samples were obtained from 11 healthy human subjects, 10 liver cancer human subjects, 27 lung cancer human subjects, and 21 pancreatic cancer human subjects. DNA was extracted from the blood samples and library preparation was performed according to: (i) the ssDNA library preparation methods set forth in Example 1 and (ii) other dsDNA library preparation methods (E7120 NEBNext® enzymatic methyl-seq kit E7120).

Both the ssDNA libraries (generated by the methods disclosed herein) and the dsDNA libraries were subject to targeted methylation conversion sequencing methods as described in Examples 2-3. The raw data files were used for alignment and methylation calling to permit targeted methylation analysis for pre-identified regions of the genome for both hypermethylation model scoring and hypomethylation model scoring.

### D. Results

FIG. 6 provides a comparison of hypermethylation model scores of cfDNA extracted from the healthy/cancer-negative (NEG), liver cancer (Liver), lung cancer (Lung), and pancreatic cancer (Pancreas) samples detected using an ssDNA library preparation method as described herein versus other dsDNA library preparation methods. Hypermethylation rates of cfDNA derived from selected genomic regions distinguished liver cancer, lung cancer, and pancreatic cancer patient-derived cfDNA from cancer-negative cfDNA when processed using both the ssDNA library preparation described herein and other dsDNA library preparation methods. This example demonstrates that the ssDNA library preparation methods described herein yield equivalent methylation analysis (e.g., hypermethylation scores) as other dsDNA library preparation methods. Thus, the ssDNA library preparation workflow described herein is equivalent to end repair-dependent methods at discriminating cancer patient-derived from healthy patient-derived cfDNA samples, e.g., when using DNA hypermethylation analysis.

FIG. 8 provides a comparison of hypomethylation scores of cfDNA extracted from the healthy/cancer-negative (NEG), liver cancer (Liver), lung cancer (Lung), and pancreatic cancer (Pancreas) patient samples detected using an ssDNA library preparation method as described herein versus a dsDNA library preparation method. As shown in FIG. 8, hypomethylation rates of cfDNA derived from selected genomic regions distinguished liver cancer, lung cancer, and pancreatic cancer patient-derived cfDNA from cancer-negative cfDNA when processed using ssDNA library preparation but not when processed using dsDNA library preparation. This example provides additional evidence that the ssDNA library preparation method described herein optimizes the recovery, methylation analysis, quantification, and target capture across multiple cancer types which further provides that this workflow can outperform end repair-dependent methods at discriminating cancer patient-derived from healthy patient-derived cfDNA samples, e.g., using DNA hypomethylation analysis.

While preferred embodiments of the present inventive concepts have been shown and described herein, it will be obvious to those skilled in the art that such embodiments are provided by way of example only. It is not intended that the inventive concepts be limited by the specific examples provided within the specification.

While the inventive concepts have been described with reference to the aforementioned specification, the descriptions and illustrations of the embodiments herein are not meant to be construed in a limiting sense. Numerous variations, changes, and substitutions will now occur to those skilled in the art without departing from the inventive concepts. Furthermore, it shall be understood that all aspects of the inventive concepts are not limited to the specific depictions, configurations or relative proportions set forth herein which depend upon a variety of conditions and variables.

It should be understood that various alternatives to the embodiments of the inventive concepts described herein may be employed in practicing the inventive concepts. It is therefore contemplated that the inventive concepts shall also cover any such alternatives, modifications, variations, or equivalents. It is intended that the following claims define the scope of the inventive concepts and that methods and structures within the scope of these claims and their equivalents be covered thereby.

1-117. (canceled)

118. A method of preparing a sequencing library for methylation sequencing of a nucleic acid molecule, comprising:
(a) obtaining a nucleic acid composition, wherein the nucleic acid composition comprises a plurality of single-stranded nucleic acid molecules obtained or derived from a biological sample;
(b) ligating a nucleic acid adapter to a single-stranded nucleic acid molecule of the plurality of single-stranded nucleic acid molecules or a derivative thereof to generate an adapter-ligated nucleic acid molecule, wherein the nucleic acid adapter comprises nucleic acids that are resistant to base conversion by a methylation enrichment method; and
(c) subjecting the adapter-ligated nucleic acid molecule or a derivative thereof to conditions sufficient to convert unmethylated cytosines to uracils using the methylation enrichment method, thereby generating a converted adapter-ligated nucleic acid molecule.

119. The method of claim 118, wherein the ligating further comprises treating with a deoxyribonucleic acid (DNA) ligase or a polynucleotide kinase.

120. The method of claim 118, wherein the nucleic acid adapter comprises a double-stranded oligonucleotide comprising an adapter sequence and an overhang sequence.

121. The method of claim 120, wherein the overhang sequence is a 3′ overhang sequence.

122. The method of claim 120, wherein the overhang sequence comprises a random sequence of oligonucleotides.

123. The method of claim 118, wherein the nucleic acid adapter comprises one or more methylated cytosine bases.

124. The method of claim 118, further comprising, prior to (a), processing the biological sample to generate the plurality of single-stranded nucleic acid molecules.

125. The method of claim 124, wherein the processing comprises denaturing a double-stranded nucleic acid molecule in the biological sample, wherein the denaturing comprises applying heat to the double-stranded nucleic acid molecule.

126. The method of claim 118, further comprising treating the single-stranded nucleic acid molecule or derivative thereof with a binding agent configured to reduce a likelihood of formation of nucleic acid duplexes, wherein the binding agent is a single-stranded nucleic acid binding protein (SSB).

127. The method of claim 118, further comprising amplifying the converted adapter-ligated nucleic acid molecule or a derivative thereof, wherein the amplifying comprises polymerase chain reaction (PCR).

128. The method of claim 118, further comprising contacting the converted adapter-ligated nucleic acid molecule or a derivative thereof with nucleic acid probes to generate an enriched nucleic acid molecule, wherein the nucleic acid probes comprise a nucleic acid sequence that is at least partially complementary to CpG or CH loci of a reference panel.

129. The method of claim 128, wherein the nucleic acid probes comprise unmethylated nucleic acid probes.

130. The method of claim 128, wherein the nucleic acid probes are configured to selectively hybridize to one or more target regions of interest that correspond to unmethylated cytosine bases at a CpG locus from the CpG or CH loci of the reference panel.

131. The method of claim 128, wherein the nucleic acid probes are configured to selectively hybridize to one or more target regions of interest that correspond to methylated cytosine bases at a CpG locus from the CpG or CH loci of the reference panel.

132. The method of claim 128, further comprising sequencing the enriched nucleic acid molecule or a derivative thereof to generate sequencing data.

133. The method of claim 132, further comprising analyzing the sequencing data to generate a methylation profile of the nucleic acid molecule of the biological sample, wherein the analyzing further comprises comparing the sequencing data to a reference sequence.

134. The method of claim 118, wherein the nucleic acid molecule is deoxyribonucleic acid (DNA).

135. The method of claim 118, wherein the biological sample is a cell-free biological sample.

136. The method of claim 118, wherein the methylation enrichment method comprises treatment with a ten eleven translocation (TET) enzyme.

137. The method of claim 118, wherein the methylation enrichment method does not comprise treatment with bisulfite.
